(19) United States
(12) Patent Application Publication
(10) Pub. No.: US 2020/0030304 A1
(43) Pub. Date: Jan. 30, 2020

(54) MITIGATION OF CNS DISORDERS BY COMBINATION THERAPY USING NEUROSTEROIDS, AND AMPA BLOCKERS

(71) Applicant: The Regents of the University of California, Oakland, CA (US)

(72) Inventors: Michael A. ROGAWSKI, Sacramento, CA (US); Ashish DHIR, Sacramento, CA (US)

(21) Appl. No.: 16/566,811

(22) Filed: Sep. 10, 2019

Related U.S. Application Data
(63) Continuation of application No. PCT/US2018/021839, filed on Mar. 9, 2018.
(60) Provisional application No. 62/470,203, filed on Mar. 11, 2017.

(51) Int. Cl. A61K 31/444; A61K 31/5513; A61K 31/57; A61P 25/08

(52) U.S. Cl. CPC A61K 31/444; A61K 31/5513; A61K 45/06; A61P 25/08; A61K 31/57

(57) ABSTRACT

Provided are compositions and methods for treating epilepsy, including epilepsy caused by exposure to organophosphate nerve agents, that entail co-formulation and/or co-administration of a benzodiazepine, a neurosteroid and an AMPA receptor antagonist.

### CROSS-REFERENCE TO RELATED APPLICATIONS

This application claims the benefit under 35 U.S.C. § 119(e) of U.S. Provisional Application No. 62/470,203, filed on Mar. 11, 2017, which is hereby incorporated herein by reference in its entirety for all purposes.

### STATEMENT OF GOVERNMENTAL SUPPORT

This work was supported in part by Grant No U54 NS079202 from the National Institutes of Health. The Government has certain rights in this invention.

### BACKGROUND

Status epilepticus is a major cause of mortality associated with organophosphates (OP)-induced poisoning.

Benzodiazepines are the current standard of care treatment available for managing OP-induced status epilepticus. However, these agents are found to provide inadequate control over seizures, especially when administered at a delayed time.

Allopregnanolone is a neurosteroid agent that acts as a positive allosteric modulator of both synaptic and extra-synaptic GABA-A receptors. Evidences have shown that allopregnanolone can act synergistically with benzodiazepines in order to provide seizure control.

### SUMMARY

In one aspect, provided are pharmaceutical compositions. In some embodiments, the compositions comprise a neurosteroid and an α amino-3-hydroxy-5-methyl-4-isoxazolepropionic acid (AMPA) receptor antagonist. In some embodiments, the composition comprises one or more of the neurosteroid and the AMPA receptor antagonist in a subtherapeutic or non-therapeutic dose. In some embodiments, the composition further comprises a benzodiazepine. In some embodiments, the composition comprises the benzodiazepine in a subtherapeutic dose. In some embodiments, the composition is formulated for oral administration. In some embodiments, the composition is formulated for parenteral delivery. In some embodiments, the parenteral delivery or administration is via a route selected from the group consisting of inhalational, intrapulmonary, intramuscular, subcutaneous, transmucosal and intravenous. In some embodiments, the benzodiazepine is a positive modulator of synaptic GABA-A receptors. In some embodiments, the benzodiazepine is an agonist of the benzodiazepine recognition site on GABA-A receptors and stimulates endogenous neurosteroid synthesis. In some embodiments, the benzodiazepine is selected from the group consisting of bretazenil, clonazepam, cloxazolam, clorazepate, diazepam, fludiazepam, flutoprazepam, lorazepam, midazolam, nimetazepam, nitrazepam, phenazepam, temazepam and clobazam. In some embodiments, the benzodiazepine is midazolam. In some embodiments, the neurosteroid is a positive modulator of synaptic and extrasynaptic GABA-A receptors. In some embodiments, the neurosteroid is selected from the group consisting of allopregnanolone, allotetrahydrodeoxycorticosterone, ganaxolone, alphaxolone, alphadolone, hydroxydione, minaxolone, and Althesin. In some embodiments, the neurosteroid is allopregnanolone. In some embodiments, the AMPA receptor antagonist is selected from the group consisting of perampanel, selurampanel, talampanel, tezampanel, fanapanel (a.k.a., ZK-200775), irampanel, kynurenic acid, CFM-2, CNQX, CNQX disodium salt, CP 465022 hydrochloride, DNQX, DNQX disodium salt, Evans Blue tetrasodium salt, GYKI 47261 dihydrochloride, GYKI 52466 dihydrochloride, GYKI 53655 hydrochloride, IEM 1925 dihydrobromide, Naspm trihydrochloride, NBQX, NBQX disodium salt, Philanthotoxin 74, SYM 2206, UBP 282, and YM 90K hydrochloride. In some embodiments, the AMPA receptor antagonist is a selective antagonist of an AMPA receptor. In some embodiments, the AMPA receptor antagonist is perampanel. In some embodiments, the composition comprises allopregnanolone and perampanel, and optionally, further comprises midazolam. In some embodiments, one or more of the neurosteroid and AMPA receptor antagonist, and optionally a benzodiazepine, is suspended or dissolved in an aqueous solution comprising a glycol and at least one alcohol having five or fewer carbons. In some embodiments, the glycol is selected from the group consisting of ethylene glycol, propylene glycol, and analogs and mixtures thereof. In some embodiments, the alcohol is selected from the group consisting of methanol, ethanol, propanol, isopropanol, and mixtures thereof. In some embodiments, the aqueous solution comprises a glycol:alcohol:water ratio of 7:2:1. In some embodiments, the neurosteroid (e.g., allopregnanolone) is present in a concentration from about 3 mg/mL to about 12 mg/mL, e.g., about 3, 4, 5, 6, 7, 8, 9, 10, 11, 12 mg/mL. In some embodiments, the AMPA receptor antagonist (e.g., perampanel) is present in a concentration from about 1 mg/mL to about 8 mg/mL, e.g., about 1, 2 3, 4, 5, 6, 7, 8 mg/mL. In some embodiments, one or more of the neurosteroid, the AMPA receptor antagonist, and optionally a benzodiazepine, is suspended or dissolved in a cyclodextrin or an edible oil. In some embodiments, the cyclodextrin is selected from the group consisting of an α-cyclodextrin, a β-cyclodextrin or a γ-cyclodextrin. In some embodiments, the cyclodextrin is selected from the group consisting of α-cyclodextrin; β-cyclodextrin; γ-cyclodextrin; methyl acyclodextrin; methyl β-cyclodextrin; methyl γ-cyclodextrin; ethyl βcyclodextrin; butyl α-cyclodextrin; butyl β-cyclodextrin; butyl γ-cyclodextrin; pentyl γ-cyclodextrin; hydroxyethyl β-cyclodextrin; hydroxyethyl γcyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl acyclodextrin; acetyl β-cyclodextrin; acetyl γ-cyclodextrin; propionyl pcyclodextrin; butyryl β-cyclodextrin; succinyl α-cyclodextrin; succinyl βcyclodextrin; succinyl γ-cyclodextrin; benzoyl β-cyclodextrin; palmityl βcyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl βcyclodextrin; phosphate ester α-cyclodextrin; phosphate ester β-cyclodextrin; phosphate ester γ-cyclodextrin; 3-trimethylammonium-2-hydroxypropyl pcyclodextrin; sulfobutyl ether β-cyclodextrin; carboxymethyl α-cyclodextrin; carboxymethyl β-cyclodextrin; carboxymethyl γ-cyclodextrin, alkyl cyclodextrins, hydroxy alkyl cyclodextrins, carboxy alkyl cyclodextrins and sulfoalkyl ether cyclodextrins, and combinations thereof. In some embodiments, the edible oil comprises one or more vegetable oils. In some embodiments, the vegetable oil is selected from the group consisting of coconut oil, corn oil, cottonseed oil, olive oil, palm oil, peanut oil, rapeseed oil, canola oil, safflower oil, sesame oil, soybean oil, sunflower oil, and mixtures thereof. In some embodiments, the edible oil is canola oil. In some embodiments, the edible oil comprises one or more nut oils. In some embodiments, the nut oil is selected from the group consisting of almond oil, cashew oil, hazelnut oil, macadamia oil, mongongo nut oil, pecan oil, pine nut oil, pistachio oil, walnut oil, and mixtures thereof. In some embodiments, the composition is contained within a soft gel capsule.

In a further aspect, provided are methods of preventing or terminating a seizure in a subject in need thereof. In another aspect, provided are methods of accelerating the termination or abortion of an impending seizure in a subject in need thereof. In another aspect, provided are methods of preventing, treating, reversing, reducing, mitigating and/or ameliorating one or more symptoms associated with a mood disorder or depression in a subject in need thereof. In some embodiments, the methods comprise administration to the subject of an effective amount of a composition, as described above and herein.

In a further aspect, provided are methods of preventing or terminating a seizure in a subject in need thereof. In another aspect, provided are methods of accelerating the termination or abortion of an impending seizure in a subject in need thereof. In some embodiments, the methods comprise co-administration to the subject of an effective amount of a neurosteroid and an AMPA receptor antagonist. In some embodiments, the subject has been exposed to or is at risk of being exposed an organophosphate nerve agent. In some embodiments, the subject is experiencing aura. In some embodiments, the subject has been warned of an impending seizure. In some embodiments, the subject is experiencing a seizure. In some embodiments, the subject has status epilepticus. In some embodiments, the subject has refractory status epilepticus. In some embodiments, the subject has super refractory status epilepticus. In some embodiments, the subject has myoclonic epilepsy. In some embodiments, the subject suffers from seizure clusters. In some embodiments, the seizure is a tonic seizure. In some embodiments, the seizure is a clonic seizure. In some embodiments, the seizure or impending seizure is terminated or aborted within 5 minutes of co-administration of the neurosteroid, the AMPA receptor antagonist, and optionally a benzodiazepine.

In a further aspect, provided are methods of preventing, treating, reversing, reducing, mitigating and/or ameliorating one or more symptoms associated with mood disorder or depression in a subject in need thereof. In some embodiments, the methods comprise co-administration to the subject of an effective amount of a neurosteroid and an AMPA receptor antagonist. In some embodiments, the mood disorder or depression is selected from clinical depression, postnatal or postpartum depression, atypical depression, melancholic depression, major depressive disorder (MDD), Psychotic Major Depression (PMD), catatonic depression, Seasonal Affective Disorder (SAD), dysthymia, double depression, Depressive Personality Disorder (DPD), Recurrent Brief Depression (RBD), minor depressive disorder, bipolar disorder or manic depressive disorder, post-traumatic stress disorders, depression caused by chronic medical conditions, treatment-resistant depression, refractory depression, suicidality, suicidal ideation, or suicidal behavior.

With respect to further embodiments of the methods, in some embodiments, the methods comprise co-administration of a benzodiazepine. In some embodiments, the neurosteroid and the AMPA receptor antagonist, and optionally the benzodiazepine, are co-administered together and/or by the same route of administration. In some embodiments, the neurosteroid and the AMPA receptor antagonist, and optionally the benzodiazepine, are co-administered separately and/or by different routes of administration. In some embodiments, one or more of the neurosteroid and the AMPA receptor antagonist, and optionally the benzodiazepine, are co-administered in a subtherapeutic or non-therapeutic dose. In some embodiments, one or more of the neurosteroid and the AMPA receptor antagonist, and optionally the benzodiazepine, is administered orally. In some embodiments, one or more of the neurosteroid and the AMPA receptor antagonist, and optionally the benzodiazepine, is administered parenterally. In some embodiments, the parenteral delivery or administration is via a route selected from the group consisting of inhalational, intrapulmonary, intramuscular, subcutaneous, transmucosal and intravenous. In some embodiments, one or more of the neurosteroid and the AMPA receptor antagonist, and optionally the benzodiazepine, is administered once. In some embodiments, one or more of the neurosteroid and the AMPA receptor antagonist, and optionally the benzodiazepine, is administered multiple times. In some embodiments, the benzodiazepine is a positive modulator of synaptic GABA-A receptors. In some embodiments, the benzodiazepine is an agonist of the benzodiazepine recognition site on GABA-A receptors and stimulates endogenous neurosteroid synthesis. In some embodiments, the benzodiazepine is selected from the group consisting of bretazenil, clonazepam, cloxazolam, clorazepate, diazepam, fludiazepam, flutoprazepam, lorazepam, midazolam, nimetazepam, nitrazepam, phenazepam, temazepam and clobazam. In some embodiments, the benzodiazepine is midazolam. In some embodiments, the benzodiazepine is administered at a dose between about 0.5 mg/kg to about 4.0 mg/kg. In some embodiments, the neurosteroid is a positive modulator of synaptic and extrasynaptic GABA-A receptors. In some embodiments, the neurosteroid is selected from the group consisting of allopregnanolone, allotetrahydrodeoxycorticosterone, ganaxolone, alphaxolone, alphadolone, hydroxydione, minaxolone, and Althesin. In some embodiments, the neurosteroid is administered in a therapeutically effective two-level dosing regimen comprising a first hourly infusion of a higher loading dose, followed by a second hourly infusion of a lower maintenance dose. In some embodiments, the neurosteroid is administered in a loading dose infusion administered over 1 hour followed by a maintenance dose infusion for the next 95 hours, followed by tapered or lowered doses to discontinue treatment. In some embodiments, the neurosteroid is administered in a therapeutically effective pyramid dosing regimen comprising a first ramp-up or step-up or increasing hourly dose infusion to a achieve a maintenance serum concentration, followed by a second hourly infusion of a constant maintenance dose, followed by a third step-down or tapering or decreasing hourly dose infusion to wean the patient or discontinue treatment. In some embodiments, the neurosteroid is administered over a period of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or more. In some embodiments, the neurosteroid is administered in a solution having a concentration of neurosteroid between about 0.25 mg/mL and about 15 mg/mL. In some embodiments, the neurosteroid is administered in a solution having a concentration of neurosteroid between about 1.0 mg/mL and about 2.0 mg/mL, e.g., about 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL or 2.0 mg/mL. In some embodiments, the dosage of neurosteroid administered by infusion produces a steady-state serum neurosteroid concentration between about 50 nM and about 2500 nM. In some embodiments, the neurosteroid is administered at a dose between about 1 mg/kg to about 10 mg/kg. In some embodiments, the neurosteroid is formulated in a solution comprising at least 6% sulfobutylether-β-cyclodextrin (SBEβCD), e.g., at least about 12%, 15%, 20%, 25% or 30% SBEβCD. In some embodiments, the neurosteroid is formulated in a buffered solution. In some embodiments, the AMPA receptor antagonist is selected from the group consisting of perampanel, selurampanel, talampanel, tezampanel, fanapanel (a.k.a., ZK-200775), irampanel, kynurenic acid, CFM-2, CNQX, CNQX disodium salt, CP 465022 hydrochloride, DNQX, DNQX disodium salt, Evans Blue tetrasodium salt, GYKI 47261 dihydrochloride, GYKI 52466 dihydrochloride, GYKI 53655 hydrochloride, IEM 1925 dihydrobromide, Naspm trihydrochloride, NBQX, NBQX disodium salt, Philanthotoxin 74, SYM 2206, UBP 282, and YM 90K hydrochloride. In some embodiments, the AMPA receptor antagonist is administered at a dose between about 0.5 mg/kg to about 4.0 mg/kg. In some embodiments, one or more of the neurosteroid and the AMPA receptor antagonist, and optionally the benzodiazepine, is self-administered by the subject. In some embodiments, one or more of the neurosteroid and the AMPA receptor antagonist, and optionally the benzodiazepine, is administered via intramuscular, inhalational or intrapulmonary administration. In some embodiments, one or more of the neurosteroid and the AMPA receptor antagonist, and optionally the benzodiazepine, is administered via inhalational or intrapulmonary administration and is nebulized. In some embodiments, one or more of the neurosteroid and the AMPA receptor antagonist, and optionally the benzodiazepine, is not heated prior to administration. In some embodiments, the nebulized particles are about 3 μm or smaller. In some embodiments, the nebulized particles are about 2 3 μm. In some embodiments, one or more of the neurosteroid and the AMPA receptor antagonist, and optionally the benzodiazepine, is delivered to the distal alveoli. In some embodiments, the subject is human.

### Definitions

As used herein, “administering” refers to local and systemic administration, e.g., including enteral, parenteral, pulmonary, and topical/transdermal administration. Routes of administration for the agents (e.g., a neurosteroid, an AMPA receptor antagonist, and optionally a benzodiazepine) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administration” refers to the presence of all active agents in the blood at the same time. Active agents that are co-administered can be delivered concurrently (i.e., at the same time) or sequentially.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The term “effective amount” or “pharmaceutically effective amount” refer to the amount and/or dosage, and/or dosage regime of one or more compounds necessary to bring about the desired result e.g., an amount sufficient prevent, abort or terminate a seizure.

“Sub-therapeutic dose” refers to a dose of a pharmacologically active agent(s), either as an administered dose of pharmacologically active agent, or actual level of pharmacologically active agent in a subject that functionally is insufficient to elicit the intended pharmacological effect in itself (e.g., to abort or prevent a seizure), or that quantitatively is less than the established therapeutic dose for that particular pharmacological agent (e.g., as published in a reference consulted by a person of skill, for example, doses for a pharmacological agent published in the Physicians' Desk Reference, PDR Network; 71st 2017 ed. edition (Dec. 13, 2016), Thomson Healthcare or Brunton, et al., Goodman & Gilman's The Pharmacological Basis of Therapeutics, 13th edition, 2017, McGraw-Hill). A “sub-therapeutic dose” can be defined in relative terms (i.e., as a percentage amount (less than 100%) of the amount of pharmacologically active agent conventionally administered). For example, a sub-therapeutic dose amount can be about 1% to about 75% of the amount of pharmacologically active agent conventionally administered. In some embodiments, a sub-therapeutic dose can be less than about 75%, 50%, 30%, 25%, 20%, 10% or less, than the amount of pharmacologically active agent conventionally administered. A sub-therapeutic dose amount can be in the range of about 1% to about 75% of the amount of pharmacologically active agent known to elicit the intended pharmacological effect. In some embodiments, a sub-therapeutic dose can be less than about 75%, 50%, 30%, 25%, 20%, 10% or less, than the amount of pharmacologically active agent known to elicit the intended pharmacological effect.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease.

The terms “reduce,” “inhibit,” “relieve,” “alleviate” refer to the detectable decrease in the frequency, severity and/or duration of seizures. A reduction in the frequency, severity and/or duration of seizures can be measured by self-assessment (e.g., by reporting of the patient) or by a trained clinical observer. Determination of a reduction of the frequency, severity and/or duration of seizures can be made by comparing patient status before and after treatment.

As used herein, the phrase “consisting essentially of” refers to the genera or species of active pharmaceutical agents (e.g., one or more neurosteroids, one or more AMPA receptor antagonists, and optionally one or more benzodiazepines) and excipient (e.g., a cyclodextrin, an edible oil) included in a method or composition. In various embodiments, other unmentioned or unrecited active ingredients and inactive are expressly excluded. In various embodiments, additives (e.g., surfactants, acids (organic or fatty), alcohols, esters, co-solvents, solubilizers, lipids, polymers, glycols) are expressly excluded.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other healthworker in a hospital, psychiatric care facility, as an outpatient, or other clinical context. In certain embodiments the subject may not be under the care or prescription of a physician or other healthworker.

The term “edible oil” refers to an oil that is digestible by a mammal. Preferred oils are edible or digestible without inducing undesirable side effects.

The term “neuroactive steroid” or “neurosteroid” refers to steroid compounds that rapidly alter neuronal excitability through interaction with neurotransmitter-gated ion channels. Neurosteroids act as allosteric modulators of neurotransmitter receptors, such as GABA_{A}, NMDA, and sigma receptors. Neurosteroids find use as sedatives for the purpose of general anaesthesia for carrying out surgical procedures, and in the treatment of epilepsy and traumatic brain injury. Illustrative neurosteroids include, e.g., allopregnanolone, Ganaxolone, alphaxolone, alphadolone, hydroxydione, minaxolone, and Althesin (a mixture of alphaxolone and alphadolone).

### BRIEF DESCRIPTION OF THE DRAWINGS

FIG. 1 illustrates the chemical structures of a representative benzodiazepine (midazolam), a representative neurosteroid (allopregnanolone), and a representative AMPA receptor antagonist.

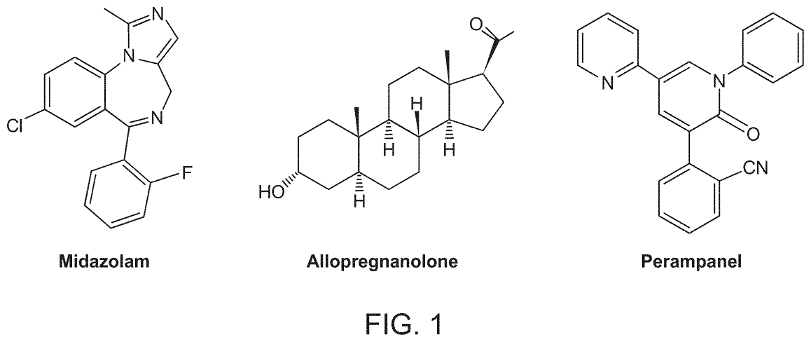

FIG. 2 illustrates a representative treatment schedule.

FIG. 3 illustrates representative EEGs.

FIG. 4 illustrates the effect of midazolam alone versus midazolam followed by allopregnanolone (dual therapy) or allopregnanolone/perampanel (triple therapy) on DFP-induced status epilepticus. EEG root mean square (RMS) amplitude was calculated in 1 min epochs and normalized to the RMS values at 0 min. Anti-seizure treatments were administered 40 min after DFP injection; the time of the treatment is designated 0 min. Error bars are not shown in the graph for clarity. Vehicle-treated animals exhibit continuous seizures but the RMS amplitude diminishes over the 5 h recording period. Midazolam (1.8 mg/kg, IM) reduces the RMS EEG amplitude but spikes and seizure discharges are not terminated. Both dual or triple therapy were effective in rapidly reducing the RMS EEG amplitude. Treatment with triple therapy however brought back the RMS EEG amplitude to the normal basal levels. Data points represent the mean±S.E.M. of normalized RMS values from experiments with 6 rats.

FIG. 5 illustrates the effect of midazolam alone versus midazolam followed by allopregnanolone (dual therapy) or allopregnanolone/perampanel (triple therapy) on behavioral seizure scores induced by DFP administration. Anti-seizure treatments were administered 40 min after DFP injection; the time of the treatment is designated 40 min. Error bars are not shown in the graph for clarity. Midazolam (1.8 mg/kg, IM) was in-effective in reducing the behavioral seizure scores caused due to DFP. Dual therapy consisting of midazolam (1.8 mg/kg) and allopregnanolone (6 mg/kg) normalized the behavioral seizure scores in 83.33% of animals. However, the seizure scores become normal with triple therapy in 100% of animals tested.

FIG. 6 illustrates loss of righting reflex in rats that were administered with midazolam (1.8 mg/kg, IM), allopregnanolone (6 mg/kg, IM) and perampanel (2 mg/kg, IM) at sequences of 10 min.

FIG. 7 illustrates a treatment paradigm in a diisopropyl fluorophosphates (DFP)-induced status epilepticus animal model.

FIGS. 8A-B. A. Comparison of the effect a single intramuscular injection of the combination of allopregnanolone (6 mg/kg) and perampanel (2 mg/kg) administered along with standard of care midazolam (1.8 mg/kg, IM) with the effect of standard of care midazolam (1.8 mg/kg, IM) alone on DFP-induced status epilepticus. Top panel A shows representative EEG recordings and bottom panel B depicts calculated normalized RMS amplitude. The red solid line on the top-EEG panel represents the treatment time. B. EEG root mean square (RMS) amplitude was calculated in 1 min epochs and normalized to the RMS values at 0 min. Anti-seizure treatments were administered 40 min after DFP injection; the time of the treatment is designated 0 min. Data points represent the mean±S.E.M. of normalized RMS values from experiments with 2-6 rats. Midazolam alone was ineffective in stopping status epilepticus in this animal model. The RMS amplitude continues to be higher than baseline throughout the recording period. Treatment with a single injection of allopregnanolone and perampanel mixture along with standard of care midazolam provided rapid and complete relief from status epilepticus. Yellow dashed line in B indicates the normal RMS amplitude in awake, behaving animals as assessed by the normalized EEG RMS amplitude level at the time of DFP treatment.

FIGS. 9A-B demonstrate that the combination of allopregnanolone and perampanel terminates EEG epileptiform activity in the rat DFP status epilepticus model whereas the combination of allopregnanolone and carbamazepine (a standard sodium channel blocking anti-seizure drug) does not terminate EEG epileptiform activity in the model. Panel A, EEG recording from an experiment in which allopregnanolone (6 mg/kg, IM) and carbamazepine (30 mg/kg, IM) were administered with standard of care midazolam (1.8 mg/kg, IM). The treatment reduces the EEG amplitude but does not eliminate epileptiform (spike) activity indicating that there is not complete suppression of status epilepticus. Panel B, EEG recording from an experiment in which allopregnanolone (6 mg/kg, IM) and perampanel (2 mg/kg, IM) were administered with standard of care midazolam (1.8 mg/kg, IM). The treatment eliminates epileptiform activity as no spikes are observed indicating that there is complete suppression of status epilepticus. Graphs below show mean±S.E.M. normalized EEG root mean square (RMS) amplitude values from experiments with 5-6 rats. The graph on the right shows the data from the full 250 min recording period whereas the graph on the left shows the data from the first 100 min of recording on an expanded time scale. EEG RMS amplitude was calculated in 1 min epochs and normalized to the RMS values at 0 min. Anti-seizure treatments were administered 40 min after DFP injection; the time of the treatment is designated 0 min. Treatment with allopregnanolone and carbamazepine with standard of care midazolam rapidly reduced the mean RMS EEG amplitude but did not bring the mean RMS EEG amplitude to the baseline value prior to the onset of status epilepticus. In contrast, treatment with allopregnanolone and perampanel with standard of care midazolam caused the RMS EEG amplitude to fall to normal basal levels in animals not experiencing status epilepticus. Yellow arrowhead indicates the mean RMS amplitude level in awake, behaving animals not experiencing status epilepticus as assessed by the mean RMS amplitude value at the time of DFP treatment.

FIGS. 10A-B. A. Effect of sodium valproate (200 mg/kg, IP) vs. allopregnanolone+perampanel combination therapy [allopregnanolone (6 mg/kg., IM)+perampanel (2 mg/kg, IM)] with standard of care midazolam (1.8 mg/kg, IM) on DFP-induced status epilepticus. Top Panel shows representative EEG recordings and bottom panel depicts calculated normalized RMS amplitude. The red solid line on the top-panel represents the treatment time. EEG root mean square (RMS) amplitude was calculated in 1 min epochs and normalized to the RMS values at 0 min. Anti-seizure treatments were administered 40 min after DFP injection; the time of the treatment is designated 0 min. B. Treatment strategy with sodium valproate on top of midazolam standard-of-care was effective in rapidly reducing the RMS EEG amplitude; however it never reached the normal baseline. Treatment with allopregnanolone with perampanel on top of midazolam therapy, however, brought back the RMS EEG amplitude to the normal basal levels (yellow arrowhead indicates the normal RMS amplitude in awake, behaving animals as assessed by the level at the time of DFP treatment, which is 16.4% the value during full blown seizure activity at 40 min). Data points represent the mean±S.E.M. of normalized RMS values from experiments with 6 rats.

FIG. 11 illustrates a comparison of the effect of perampanel (2 mg/kg, IM) and standard of care midazolam (1.8 mg/kg, IM) with standard of care midazolam alone on DFP-induced status epilepticus rats. EEG root mean square (RMS) amplitude was calculated in 1 min epochs and normalized to the RMS values at 0 min. Anti-seizure treatments were administered 40 min after DFP injection; the time of the treatment is designated 0 min. Midazolam failed to terminate EEG status epilepticus in this animal model. Treatment with perampanel and midazolam reduced the RMS EEG amplitude with respect to midazolam alone. However, the onset of action of perampanel was slow (compare with onset of action of the combination of allopregnanolone and perampanel in FIGS. 4, 8 and 9). EEG status epilepticus was not terminated until about 90 min after perampanel administration. The yellow dashed line indicates the normal RMS amplitude in awake, behaving animals as assessed by the level at the time of DFP treatment. Data points represent the mean±S.E.M. of normalized RMS values from experiments with 6 rats. This experiments demonstrates that the combination of allopregnanolone and perampanel is required to obtain rapid termination of status epilepticus.

### DETAILED DESCRIPTION

1. Introduction

There is no adequate treatment for organophosphate nerve agent-induced status epilepticus. Current standard-of-care emergency treatment is with a benzodiazepine such as midazolam, which may be administered in the field by autoinjector or in the emergency department by IV infusion. Oftentimes, status epilepticus is refractory to benzodiazepines. We have discovered that the combination of a neurosteroid that is a positive modulator of synaptic and extrasynaptic GABA-A receptors (e.g., allopregnanolone), an AMPA receptor antagonist (e.g., perampanel), and optionally, a benzodiazepine that is a positive modulator of synaptic GABA-A receptors (e.g., midazolam), is highly effective in treating organophosphate nerve agent induced status epilepticus. The AMPA receptor antagonist perampanel is an antiseizure drug commonly used in the treatment of epilepsy. Perampanel is not known to be superior to other antiseizure agents, such as carbamazepine, in the treatment of focal epilepsy in humans. Surprisingly and unexpectedly, we found that the combination of perampanel and allopregnanolone was superior to the combination of carbamazepine and allopregnanolone in the treatment of status epilepticus. Specifically, the combination of perampanel and allopregnanolone administered with standard of care midazolam terminated epileptiform activity in a rat model of DFP-induced status epilepticus whereas the combination of carbamazepine and allopregnanolone also administered with standard of care midazolam did not terminate epileptiform activity, indicating that the latter combination did not fully treat the status epilepticus.

Because OP seizure are believed to be dependent in part on glutamate-induced excitation, we studied the triple combination with perampanel, an antagonist of AMPA-type ionotropic glutamate receptors. SE was induced in male Sprague-Dawley rats with a SQ injection of 4 mg/kg DFP. Atropine (2 mg/kg) and 2-PAM (pralidoxime; 25 mg/kg) were administered IM 1 min after DFP. Continuous video-EEG monitoring was carried out from permanently implanted cortical electrodes before and for at least 5 hours following the DFP injection. Combination therapies with midazolam (1.8 mg/kg), allopregnanolone (6 mg/kg), and perampanel (2 mg/kg) were administered IM 40 min after the DFP injection. DFP induced a robust SE within minutes of injection, which is associated with high mortality. Administration of midazolam (1.8 mg/kg) alone was ineffective in terminating SE. The dual combination of allopregnanolone and midazolam terminated behavioral seizures and resulted in persistent normalization of the EEG in 83% of animals; the remaining animals had continuous spikes and ictal activity following a brief termination of SE. Triple therapy with midazolam, allopregnanolone and perampanel stopped continuous behavioral and EEG seizure activity in 100% of animals within a few min of administration. Sedation was observed with both the dual and triple combinations. There was no mortality in any of the treated groups. The double and especially the triple therapy were highly effective not only in preventing lethality but also in suppressing EEG seizure activity. We conclude that the double and triple combinations are a promising approach to the treatment of OP-induced SE.

2. Conditions Amenable to Treatment

a. Seizure/Convulsive Disorders

Co-administration of a neurosteroid, an AMPA receptor antagonist, and optionally a benzodiazepine, finds use in the rapid amelioration and/or termination of seizures. In various embodiments, the seizures may be due to an epileptic condition.

The term “epilepsy” refers to a chronic neurological disorder characterized by recurrent unprovoked seizures. These seizures are transient signs and/or symptoms of abnormal, excessive or synchronous neuronal activity in the brain. There are over 40 different types of epilepsy, including without limitation childhood absence epilepsy, juvenile absence epilepsy, benign Rolandic epilepsy, clonic seizures, complex partial seizures, frontal lobe epilepsy, febrile seizures, infantile spasms, juvenile myoclonic epilepsy, Lennox-Gastaut syndrome, Landau-Kleffner Syndrome, myoclonic seizures, mitochondrial disorders associated with seizures, Lafora Disease, progressive myoclonic epilepsies, reflex epilepsy, and Rasmussen's syndrome. There are also numerous types of seizures including simple partial seizures, complex partial seizures, generalized seizures, secondarily generalized seizures, temporal lobe seizures, tonic-clonic seizures, tonic seizures, psychomotor seizures, limbic seizures, status epilepticus, refractory status epilepticus or super refractory status epilepticus, abdominal seizures, akinetic seizures, autonomic seizures, massive bilateral myoclonus, drop seizures, focal seizures, gelastic seizures, Jacksonian march, motor seizures, multifocal seizures, neonatal seizures, nocturnal seizures, photosensitive seizure, sensory seizures, sylvan seizures, withdrawal seizures and visual reflex seizures.

The most widespread classification of the epilepsies divides epilepsy syndromes by location or distribution of seizures (as revealed by the appearance of the seizures and by EEG) and by cause. Syndromes are divided into localization-related epilepsies, generalized epilepsies, or epilepsies of unknown localization. Localization-related epilepsies, sometimes termed partial or focal epilepsies, arise from an epileptic focus, a small portion of the brain that serves as the irritant driving the epileptic response. Generalized epilepsies, in contrast, arise from many independent foci (multifocal epilepsies) or from epileptic circuits that involve the whole brain. Epilepsies of unknown localization remain unclear whether they arise from a portion of the brain or from more widespread circuits.

Epilepsy syndromes are further divided by presumptive cause: idiopathic, symptomatic, and cryptogenic. Idiopathic epilepsies are generally thought to arise from genetic abnormalities that lead to alterations in brain excitability. Symptomatic epilepsies arise from the effects of an epileptic lesion, whether that lesion is focal, such as a tumor, or a defect in metabolism causing widespread injury to the brain. Cryptogenic epilepsies involve a presumptive lesion that is otherwise difficult or impossible to uncover during evaluation. Forms of epilepsy are well characterized and reviewed, e.g., in Epilepsy: A Comprehensive Textbook (3-volume set), Engel, et al., editors, 2nd Edition, 2007, Lippincott, Williams and Wilkins; and The Treatment of Epilepsy: Principles and Practice, Wyllie, et al., editors, 4th Edition, 2005, Lippincott, Williams and Wilkins; and Browne and Holmes, Handbook of Epilepsy, 4th Edition, 2008, Lippincott, Williams and Wilkins.

In various embodiments, the patient may be experiencing an electrographic or behavioral seizure or may be experiencing a seizure aura, which itself is a localized seizure that may spread and become a full blown behavioral seizure. For example, the subject may be experiencing aura that alerts of the impending onset of a seizure or seizure cluster.

Alternatively, the subject may be using a seizure prediction device that alerts of the impending onset of a seizure or seizure cluster. Implantable seizure prediction devices are known in the art and described, e.g., in D'Alessandro, et al., IEEE TRANSACTIONS ON BIOMEDICAL ENGINEERING, VOL. 50, NO. 5, May 2003, and U.S. Patent Publication Nos. 2010/0198098, 2010/0168603, 2009/0062682, and 2008/0243022.

The subject may have a personal or familial history of any of the epileptic conditions and other conditions amenable to treatment described herein. The subject may have been diagnosed as having any of the conditions amenable to treatment, e.g., epileptic conditions described herein. In some embodiments, the subject has or is at risk of suffering the condition amenable to treatment, e.g., status epilepticus, convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; periodic lateralized epileptiform discharges. In some embodiments, the seizure/convulsive disorder is a traumatic brain injury. In some embodiments, the seizure/convulsive disorder is a seizure, e.g., acute repetitive seizures, cluster seizures. In some embodiments, the subject has or is at risk of suffering a myoclonic seizure or myoclonic epilepsy, e.g., juvenile myoclonic epilepsy. The PTZ seizure model demonstrated herein is predictive of utility and/or activity in counteracting myoclonic seizures or myoclonic epilepsy in humans.

In various embodiments, the subject may be at risk of exposure to or may have been exposed to tetramethylenedisulfotetramine (TETS).

In various embodiments, the subject may be at risk of exposure to or may have been exposed to a nerve agent or a pesticide that can cause seizures. Illustrative nerve agents that can cause seizures include, e.g., organophosphorus nerve agents, e.g., diisopropylfluorophosphate (DFP), tabun, sarin, soman, GF, VR and/or VX. Illustrative pesticides that can cause seizures include, e.g., organophosphate pesticides (e.g., Acephate (Orthene), Azinphos-methyl (Gusathion, Guthion), Bensulide (Bsan, Lescosan), Bomyl (Swat), Bromophos (Nexion), Bromophos-ethyl (Nexagan), Cadusafos (Apache, Ebufos, Rugby), Carbophenothion (Trithion), Chlorethoxyfos (Fortress), Chlorfenvinphos (Apachlor, Birlane), Chlormephos (Dotan), Chlorphoxim (Baythion-C), Chlorpyrifos (Brodan, Dursban, Lorsban), Chlorthiophos (Celathion), Coumaphos (Asuntol, Co-Ral), Crotoxyphos (Ciodrin, Cypona), Crufomate (Ruelene), Cyanofenphos (Surecide), Cyanophos (Cyanox), Cythioate (Cyflee, Proban), DEF (De-Green), E-Z-Off D), Demeton (Systox), Demeton-S-methyl (Duratox, Metasystoxl), Dialifor (Torak), Diazinon, Dichlorofenthion, (VC-13 Nemacide), Dichlorvos (DDVP, Vapona), Dicrotophos (Bidrin), Dimefos (Hanane, Pestox XIV), Dimethoate (Cygon, DeFend), Dioxathion (Delnav), Disulfoton (Disyston), Ditalimfos, Edifenphos, Endothion, EPBP (S-seven), EPN, Ethion (Ethanox), Ethoprop (Mocap), Ethyl parathion (E605, Parathion, thiophos), Etrimfos (Ekamet), Famphur (Bash, Bo-Ana, Famfos), Fenamiphos (Nemacur), Fenitrothion (Accothion, Agrothion, Sumithion), Fenophosphon (Agritox, trichloronate), Fensulfothion (Dasanit), Fenthion (Baytex, Entex, Tiguvon), Fonofos (Dyfonate, N-2790), Formothion (Anthio), Fosthietan (Nem-A-Tak), Heptenophos (Hostaquick), Hiometon (Ekatin), Hosalone (Zolone), IBP (Kitazin), Iodofenphos (Nuvanol-N), Isazofos (Brace, Miral, Triumph), Isofenphos (Amaze, Oftanol), Isoxathion (E-48, Karphos), Leptophos (Phosvel), Malathion (Cythion), Mephosfolan (Cytrolane), Merphos (Easy Off-D, Folex), Methamidophos (Monitor), Methidathion (Supracide, Ultracide), Methyl parathion (E601, Penncap-M), Methyl trithion, Mevinphos (Duraphos, Phosdrin), Mipafox (Isopestox, Pestox XV), Monocrotophos (Azodrin), Naled (Dibrome), Oxydemeton-methyl (Metasystox-R), Oxydeprofos (Metasystox-S), Phencapton (G 28029), Phenthoate (Dimephenthoate, Phenthoate), Phorate (Rampart, Thimet), Phosalone (Azofene, Zolone), Phosfolan (Cylan, Cyolane), Phosmet (Imidan, Prolate), Phosphamidon (Dimecron), Phostebupirim (Aztec), Phoxim (Baythion), Pirimiphos-ethyl (Primicid), Pirimiphos-methyl (Actellic), Profenofos (Curacron), Propetamphos (Safrotin), Propyl thiopyrophosphate (Aspon), Prothoate (Fac), Pyrazophos (Afugan, Curamil), Pyridaphenthion (Ofunack), Quinalphos (Bayrusil), Ronnel (Fenchlorphos, Korlan), Schradan (OMPA), Sulfotep (Bladafum, Dithione, Thiotepp), Sulprofos (Bolstar, Helothion), Temephos (Abate, Abathion), Terbufos (Contraven, Counter), Tetrachlorvinphos (Gardona, Rabon), Tetraethyl pyrophosphate (TEPP), Triazophos (Hostathion), and Trichlorfon (Dipterex, Dylox, Neguvon, Proxol).

b. CNS Conditions Related to GABA Modulation

Co-administration of a neurosteroid, an AMPA receptor antagonist, and optionally a benzodiazepine, finds use as a hormone or steroid replacement therapy in a subject. In an embodiment, a subject described herein has experienced a decrease in a steroid or hormone level prior to treatment with the combined active agents, as described herein. For example, a subject generally experiences a decrease in allopregnanolone subsequent to delivery of an infant. In an embodiment, a subject can be administered a compound described herein (e.g., a neurosteroid (e.g., allopregnanolone), an AMPA receptor antagonist (e.g., perampanel), and optionally a benzodiazepine (e.g., midazolam)) after experiencing a decrease in steroid or hormone level. In an embodiment, the decrease in hormone or steroid level in the subject in need of treatment, prior to treatment is at least by a factor of 2 (e.g., at least a factor of 3, 4, 5, 10 or 100).

Co-administration of a neurosteroid, an AMPA receptor antagonist, and optionally a benzodiazepine, finds use for the treatment and prevention of CNS-related conditions in a subject related to GABA modulation. GABA modulation, as used herein, refers to the inhibition or potentiation of GABA receptor function. Accordingly, the compounds and pharmaceutical compositions provided herein find use as therapeutics for preventing and/or treating CNS conditions in mammals including humans and non-human mammals. Thus, and as stated earlier, the present methods include within their scope, and extend to, the recited methods of treatment, as well as to the compounds for such methods, and to the use of such compounds for the preparation of medicaments useful for such methods.

Exemplary CNS conditions related to GABA-modulation include, but are not limited to, sleep disorders (e.g., insomnia), mood disorders (e.g., depression such as PND or perinatal depression, dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), compulsive disorders (e.g., obsessive compulsive disorder (OCD)), schizophrenia spectrum disorders (e.g., schizophrenia, schizoaffective disorder], disorders of memory and/or cognition (e.g., attention disorders (e.g., attention deficit hyperactivity disorder (ADHD)), dementia (e.g., Alzheimer's type dementia, Lewis body type dementia, vascular type dementia), movement disorders (e.g., Huntington's disease, Parkinson's disease), tremor, personality disorders (e.g., anti-social personality disorder, obsessive compulsive personality disorder), autism spectrum disorders (ASD) (e.g., autism, monogenetic causes of autism such as synaptophathy's, e.g., Rett syndrome, Fragile X syndrome, Angelman syndrome), pain (e.g., neuropathic pain, injury related pain syndromes, acute pain, chronic pain), traumatic brain injury (TBI), vascular diseases (e.g., stroke, ischemia, vascular malformations), substance abuse disorders and/or withdrawal syndromes (e.g., addiction to opiates, cocaine, and/or alcohol), and tinnitus.

Accordingly, co-administration of a neurosteroid, an AMPA receptor antagonist, and optionally a benzodiazepine, finds use in treating subjects suffering from or at risk of suffering from schizophrenia, depression (including post-partum depression), premenstrual dysphoric disorder, alcohol craving, nicotine craving, bipolar disorder, schizoaffective disorder, mood disorders, anxiety disorders, personality disorders, psychosis, compulsive disorders, posttraumatic stress disorder, autism spectrum disorder, dysthymia, social anxiety disorder, obsessive compulsive disorder, pain, sleep disorders, memory disorders, dementia, Alzheimer's Disease, a seizure disorder, traumatic brain injury, stroke, addictive disorders, autism, Huntington's Disease, insomnia, Parkinson's disease, withdrawal syndromes, tinnitus, or fragile X syndrome, lysosomal storage disorders (Niemann-Pick type C disease).

Co-administration of a neurosteroid, an AMPA receptor antagonist, and optionally a benzodiazepine, finds use for the treatment and prevention of a mood disorder, for example clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior.

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as) perinatal depression. In an embodiment, a subject experiencing perinatal depression is at increased risk of experiencing PND.

In various embodiments, clinical evaluation can be measured by the Clinical Global Impression-Improvement Scale (CGI-I), sedation using the Stanford Sleepiness Scale (SSS), safety and tolerability, assessed using adverse event reporting, vital sign measurement, laboratory data, ECG parameters, and suicidal ideation using the Columbia-Suicide Severity Rating Scale (C-SSRS). Depressive symptom severity, reproductive mood disorders, and sleepiness can be measured by the following clinician- and subject-rated outcome measures: Edinburgh Postnatal Depression Scale (EPDS), Reproductive Mood Disorders Visual Analogue Scale (RMD VAS), Generalized Anxiety Disorder 7-Item Scale (GAD-7), Patient Health Questionnaire (PHQ-9), and evaluation of individual subcategories of the HAM-D-17. In some embodiments, the diagnosis of PPD is determined by Structured Clinical Interview for DSM-V Axis I Disorders (SCID-I).

3. Therapeutic Agents

Generally, the compositions and methods comprise co-administering a neurosteroid, an AMPA receptor antagonist, and optionally a benzodiazepine. In varying embodiments, one or more of the neurosteroid, the AMPA antagonist, and optionally the benzodiazepine are co-administered at a sub-therapeutic dose or non-therapeutic dose or amount. The active agents can be co-administered concurrently or sequentially. The active agents can be co-administered via the same or different routes of administration. In various embodiments, the agents are co-administered in a single composition.

a. Neurosteroids

The terms “neuroactive steroid” or “neurosteroids” interchangeably refer to steroids that rapidly alter neuronal excitability through interaction with neurotransmitter-gated ion channels, specifically GABA_{A} receptors. Neuroactive steroids have a wide range of applications from sedation to treatment of epilepsy and traumatic brain injury. Neuroactive steroids act as direct agonists and allosteric positive modulators of GABA_{A} receptors. Several synthetic neuroactive steroids have been used as sedatives for the purpose of general anaesthesia for carrying out surgical procedures. Exemplary sedating neuroactive steroids include without limitation alphaxolone, alphadolone, hydroxydione and minaxolone. The neuroactive steroid ganaxolone finds use for the treatment of epilepsy. In various embodiments, the benzodiazepine or non-benzodiazepine benzodiazepine receptor agonist is co-administered with an endogenously occurring neurosteroid or other neuroactive steroid. Illustrative endogenous neuroactive steroids, e.g., allopregnanolone and tetrahydrodeoxycorticosterone find use. In some embodiments, the neurosteroid is selected from the group consisting of allopregnanolone, allotetrahydrodeoxycorticosterone, ganaxolone, alphaxolone, alphadolone, hydroxydione, minaxolone, and Althesin.

In various embodiments, the neurosteroid is allopregnanolone (ALP). Allopregnanolone, also known as 3α-hydroxy-5α-pregnan-20-one or 3α,5α-tetrahydroprogesterone, IUPAC name 1-(3-Hydroxy-10,13-dimethyl-2,3,4,5,6,7,8,9,11,12,14,15,16,17-tetradecahydro-1H-cyclopenta[a]phenanthren-17-yl)ethanone, and referenced as CAS number 516-54-1, is a prototypic neurosteroid present in the blood and also the brain. It is a metabolite of progesterone and modulator of GABA_{A} receptors. While allopregnanolone, like other GABA_{A} receptor active neurosteroids such as allotetrahydrodeoxycorticosterone (3α,21-dihydroxy-5α-pregnan-20-one; THDOC), positively modulates all GABA_{A} receptor isoforms, those isoforms containing δ-subunits exhibit greater magnitude potentiation. Allopregnanolone has pharmacological properties similar to other positive modulators of GABA_{A} receptors, including anxiolytic and anticonvulsant activity. Allopregnanolone is neuroprotective in many animal models of neurodegenerative conditions, including, e.g., Alzheimer's disease (Wang et al., Proc Natl Acad Sci USA. 2010 Apr. 6; 107(14):6498-503), cerebral edema (Limmroth et al., Br J Pharmacol. 1996 January; 117(1):99-104) and traumatic brain injury (He et al., Restor Neurol Neurosci. 2004; 22(1): 19-31; and He, et al., Exp Neurol. 2004 October; 189(2):404-12), Mood disorders (Robichaud and Debonnel, Int J Neuropsychopharmacol. 2006 April; 9(2): 191-200), Niemann-Pick type C disease (Griffin et al., Natl Med. 2004 July; 10(7):704-11) and acts as an anticonvulsant against chemically induced seizures, including the pentylenetetrazol (PTZ) model (Kokate et al., J Pharmacol Exp Ther. 1994 September; 270(3):1223-9). The chemical structure of allopregnanolone is depicted below in Formula I:

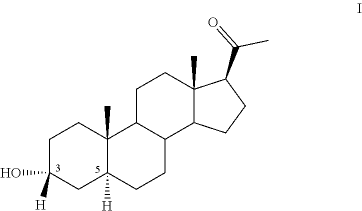 [SMILES: CC(=O)[C@H]1CCC2C3CC[C@H]4C[C@H](O)CC[C@]4(C)C3CC[C@@]21C]

In various embodiments, the compositions comprise a sulfate, salt, hemisuccinate, nitrosylated, derivative or congener of allopregnanolone.

Delivery of other neurosteroids also can be enhanced by formulation in a cyclodextrin and/or in an edible oil.

Other neurosteroids that find use in the present compositions and methods include without limitation allotetrahydrodeoxycorticosterone (3α,21-dihydroxy-5α-pregnan-20-one; THDOC), 3 α,21-dihydroxy-5b-pregnan-20-one, pregnanolone (3α-hydroxy-5β-pregnan-20-one), Ganaxolone (INN, also known as CCD-1042; IUPAC name (3α,5α)-3-hydroxy-5-methylpregnan-20-one; 1-[(3R,5 S,8R,9S,10S,13S,14S,17S)-3-hydroxy-3,10,13-trimethyl-1,2,4,5,6,7,8,9,11,12,14,15,16,17-tetradecahydrocyclopenta[a]phenanthren-17-yl]ethanone), alphaxolone, alphadolone, hydroxydione, minaxolone, and Althesin (a mixture of alphaxolone, alphadolone, tetrahydrodeoxycorticosterone, pregnenolone, dehydroepiandrosterone (DHEA), 7-substituted benz[e]indene-3-carbonitriles (see, e.g., Hu, et al., J Med Chem. (1993) 36(24):3956-67); 7-(2-hydroxyethyl)benz[e]indene analogues (see, e.g., Han, et al., J Med Chem. (1995) 38(22):4548-56); 3 α-hydroxy-5 α-pregnan-20-one and 3 α-hydroxy-5 β-pregnan-20-one analogues (see, e.g., Han, et al., J Med Chem. (1996) 39(21):4218-32); enantiomers of dehydroepiandrosterone sulfate, pregnenolone sulfate, and (3α,5β)-3-hydroxypregnan-20-one sulfate (see, e.g., Nilsson, et al., J Med Chem. (1998) 41(14):2604-13); 13,24-cyclo-18,21-dinorcholane analogues (see, e.g., Jiang, et al., J Med Chem. (2003) 46(25):5334-48); N-acylated 17α-aza-D-homosteroid analogues (see, e.g., Covey, et al., J Med Chem. (2000) 43(17):3201-4); 5 β-methyl-3-ketosteroid analogues (see, e.g., Zeng, et al., J Org Chem. (2000) 65(7):2264-6); 18-norandrostan-17-one analogues (see, e.g., Jiang, et al., J Org Chem. (2000) 65(11):3555-7); (3α,5α)- and (3α,5β)-3-hydroxypregnan-20-one analogs (see, e.g., Zeng, et al., J Med Chem. (2005) 48(8):3051-9); benz[f]indenes (see, e.g., Scaglione, et al., J Med Chem. (2006) 49(15):4595-605); enantiomers of androgens (see, e.g., Katona, et al., Eur J Med Chem. (2008) 43(1): 107-13); cyclopenta[b]phenanthrenes and cyclopenta[b]anthracenes (see, e.g., Scaglione, et al., J Med Chem. (2008) 51(5):1309-18); 2β-hydroxygonane derivatives (see, e.g., Wang, et al., Tetrahedron (2007) 63(33):7977-7984); Δ16-alphaxalone and corresponding 17-carbonitrile analogues (see, e.g., Bandyopadhyaya, et al., Bioorg Med Chem Lett. (2010) 20(22):6680-4); Δ(16) and Δ(17(20)) analogues of Δ(16)-alphaxalone (see, e.g., Stastna, et al., J Med Chem. (2011) 54(11):3926-34); neurosteroid analogs developed by CoCensys (now Purdue Neuroscience) (e.g., CCD-3693, Co2-6749 (a.k.a., GMA-839 and WAY-141839) or Sage Therapeutics (e.g., SAGE-217, SAGE-547); neurosteroid analogs described in U.S. Pat. No. 7,781,421; U.S. Patent Publication Nos. US2015/0158903 and US2015/0175651; and in PCT Patent Publications WO 2008/157460; WO 1993/003732; WO 1993/018053; WO 1994/027608; WO 1995/021617; WO 1996/016076; WO 1996/040043; WO/2018/039378; WO/2018/039378; WO/2018/013613; WO/2018/013615; WO/2018/009867; WO/2017/193046; WO/2017/173358; WO/2017/156103; WO/2017/087864; WO/2017/007832; WO/2017/007836; WO/2017/007840; WO/2016/205721; WO/2016/164763; WO/2016/134301; WO/2016/123056; WO/2016/082789; WO/2016/061537; WO/2016/061527; WO/2016/057713; WO/2016/040322; WO/2015/195967; WO/2015/195962; WO/2015/180679; WO/2015/027227; WO/2015/010054; WO/2014/169832; WO/2014/169831; WO/2014/169836; WO/2014/169833; WO/2014/160480; WO/2014/160441; WO/2014/100228; WO/2013/188792; WO/2013/112605; WO/2013/056181; and WO/2013/036835, as well as salts, hemisuccinates, nitrosylated, sulfates and derivatives thereof. The foregoing listed patents and patent publications are hereby incorporated herein by reference in their entireties for all purposes.

Additional representative synthetic progestins of use in the present compositions and methods include, but are not limited to, substitutions at the 17-position of the progesterone ring to introduce a hydroxyl, acetyl, hydroxyl acetyl, aliphatic, nitro, or heterocyclic group, modifications to produce 17α-OH esters (i.e., 17 α-hydroxyprogesterone caproate), as well as modifications that introduce 6-methyl, 6-ene, and 6-chloro substituents onto progesterone (i.e., medroxyprogesterone acetate, megestrol acetate, and chlomadinone acetate), and which retains the biologically activity of progesterone (i.e., treats a traumatic CNS injury). Such progestin derivatives include 5-dehydroprogesterone, 6-dehydro-retroprogesterone (dydrogesterone), allopregnanolone (allopregnan-3α, or 3β-ol-20-one), ethynodiol diacetate, hydroxyprogesterone caproate (pregn-4-ene-3,20-dione, 17-(1-oxohexy)oxy); levonorgestrel, norethindrone, norethindrone acetate (19-norpregn-4-en-20-yn-3-one, 17-(acetyloxy)-,(17a)-); norethynodrel, norgestrel, pregnenolone, and megestrol acetate.

Useful progestins also include allopregnone-3α or 3β, 20α or 20β-diol (see Merck Index 258-261); allopregnane-3β,21-diol-11,20-dione; allopregnane-3 β,17α-diol-20-one; 3,20-allopregnanedione, allopregnane, 3β,11β,17α, 20β,21-pentol; allopregnane-3β,17α,20β,21-tetrol; allopregnane-3α or 3β,11β,17α,21-tetrol-20-one, allopregnane-3β,17α or 20β-triol; allopregnane-3,17α,21-triol-11,20-dione; allopregnane-3β,11β,21-triol-20-one; allopregnane-3β,17α,21-triol-20-one; allopregnane-3α or 3β-ol-20-one; pregnanediol; 3,20-pregnanedione; pregnan-3α-ol-20-one; 4-pregnene-20,21-diol-3,11-dione; 4-pregnene-11β,17α,20β,21-tetrol-3-one; 4-pregnene-17α,20β,21-triol-3,11-dione; 4-pregnene-17α,20β,21-triol-3-one, and pregnenolone methyl ether. Further progestin derivatives include esters with non-toxic organic acids such as acetic acid, benzoic acid, maleic acid, malic acid, caproic acid, and citric acid and inorganic salts such as hydrochloride, sulfate, nitrate, bicarbonate and carbonate salts. Other suitable progestins include alphaxalone, alphadolone, hydroxydione, and minaxolone.

As appropriate, the neurosteroid (e.g., allopregnanolone) may or may not be micronized. As appropriate, the neurosteroid (e.g., allopregnanolone) may or may not be enclosed in microspheres in suspension in the oil or cyclodextrin.

b. AMPA Receptor Antagonists

Competitive, non-competitive and selective AMPA receptor antagonists can find use in the compositions and methods described herein. Illustrative AMPA receptor antagonists that find use include without limitation, e.g., perampanel (CAS Number 380917-97-5), talampanel (a.k.a. GYKI 537773, LY300164; CAS Number 161832-65-1), tezampanel (a.k.a., LY-293,558, NGX-424; CAS Number 150131-78-5), selurampanel (CAS Number 912574-69-7), fanapanel (a.k.a., ZK-200775; CAS Number 161605-73-8), irampanel (CAS Number 206260-33-5), kynurenic acid (CAS Number 492-27-3), CFM-2 (CAS Number 178616-26-7), CNQX (CAS Number 115066-14-3), CNQX disodium salt (CAS Number 479347-85-8), CP 465022 hydrochloride (CAS Number 199655-36-2), DNQX (CAS Number 2379-57-9), DNQX disodium salt (CAS Number 1312992-24-7), Evans Blue tetrasodium salt (CAS Number 314-13-6), GYKI 47261 dihydrochloride (CAS Number 1217049-32-5), GYKI 52466 (CAS Number 102771-26-6), GYKI 52466 dihydrochloride (CAS Number 192065-56-8), GYKI 53655 hydrochloride (CAS Number 143692-48-2), IEM 1925 dihydrobromide (CAS Number 258282-23-4), Naspm trihydrochloride (CAS Number 1049731-36-3), NBQX (CAS Number 118876-58-7), NBQX disodium salt (CAS Number 479347-86-9), Philanthotoxin 74 (CAS Number 1227301-51-0), SYM 2206 (CAS Number 173952-44-8), UBP 282 (CAS Number 544697-47-4), and YM 90K hydrochloride (CAS Number 154164-30-4).

Additional AMPA receptor antagonists that can find use in the compositions and methods described herein are described, e.g., in U.S. Patent Publication Nos. 2012/0263791, 2014/0148441 and 2015/0344468; in Intl. Patent Publication Nos. WO2014/085153; WO2013/036224; WO2011/161249; WO2011/048150; WO2011/009951; WO1993/014067; WO1993/011115; and WO1993/010783. Further AMPA receptor antagonists that can find use are described, e.g., in Inami, et al., Bioorg Med Chem. (2015) 23(8):1788-99; Koller, et al., Bioorg Med Chem Lett. (2011) 21(11):3358-61; Orain, et al., Bioorg Med Chem Lett. (2012) 22(2):996-9.

c. Benzodiazepines

In some embodiments, the compositions and methods further comprise a benzodiazepine. Any benzodiazepine known in the art finds use in the present compositions and methods. Illustrative benzodiazepines that find use include without limitation bretazenil, clonazepam, cloxazolam, clorazepate, diazepam, fludiazepam, flutoprazepam, lorazepam, midazolam, nimetazepam, nitrazepam, phenazepam, temazepam and clobazam. In some embodiments, the benzodiazepine is midazolam. In some embodiments, the benzodiazepine is diazepam.

4. Formulation and Administration

In various embodiments, the one or more neurosteroids, one or more AMPA receptor antagonists, and optionally a benzodiazepine, are formulated for intramuscular, intravenous, subcutaneous, intrapulmonary and/or inhalational administration. In various embodiments, the one or more neurosteroids, one or more AMPA receptor antagonists, and optionally a benzodiazepine, are formulated for delivery via an inhaler. In various embodiments other routes of delivery, described herein may be appropriate.

In various embodiments, the agents (e.g., the one or more neurosteroids, one or more AMPA receptor antagonists, and optionally a benzodiazepine) are nebulized. Methods and systems for intrapulmonary delivery of agents, e.g., benzodiazepines, are known in the art and find use. Illustrative systems for aerosol delivery of benzodiazepines by inhalation are described, e.g., in U.S. Pat. Nos. 5,497,763; 5,660,166; 7,060,255; and 7,540,286; and U.S. Patent Publication Nos. 2003/0032638; and 2006/0052428, each of which are hereby incorporated herein by reference in their entirety for all purposes. Preferably, the active agents (e.g., the one or more neurosteroids, one or more AMPA receptor antagonists, and optionally a benzodiazepine) are nebulized without the input of heat.

For administration of the nebulized and/or aerosolized agents (e.g., one or more neurosteroids, one or more AMPA receptor antagonists, and optionally a benzodiazepine), the size of the aerosol particulates can be within a range appropriate for intrapulmonary delivery, particularly delivery to the distal alveoli. In various embodiments, the aerosol particulates have a mass median aerodynamic diameter (“MMAD”) of less than about 5 μm, 4 μm, 3 μm, for example, ranging from about 1 μm to about 3 μm, e.g., from about 2 μm to about 3 μm, e.g., ranging from about 0.01 μm to about 0.10 μm. Aerosols characterized by a MMAD ranging from about 1 μm to about 3 μm can deposit on alveoli walls through gravitational settling and can be absorbed into the systemic circulation, while aerosols characterized by a MMAD ranging from about 0.01 μm to 0.10 μm can also be deposited on the alveoli walls through diffusion. Aerosols characterized by a MMAD ranging from about 0.15 μm to about 1 μm are generally exhaled. Thus, in various embodiments, aerosol particulates can have a MMAD ranging from 0.01 μm to about 5 μm, for example, ranging from about 0.05 μm to about 3 μm, for example, ranging from about 1 μm to about 3 μm, for example, ranging from about 0.01 μm to about 0.1 μm. The nebulized and/or aerosolized active agents (e.g., one or more neurosteroids, one or more AMPA receptor antagonists, and optionally a benzodiazepine) can be delivered to the distal alveoli, allowing for rapid absorption and efficacy.

In various embodiments, the agents (e.g., one or more neurosteroids, one or more AMPA receptor antagonists, and optionally a benzodiazepine) are formulated in a solution comprising excipients suitable for aerosolized intrapulmonary delivery. The solution can comprise one or more pharmaceutically acceptable carriers and/or excipients. Pharmaceutically acceptable refers to approved or approvable by a regulatory agency of the Federal or a state government or listed in the U. S Pharmacopoeia or other generally recognized pharmacopoeia for use in animals, and more particularly in humans.

In various embodiments, the active agents (e.g., one or more neurosteroids, one or more AMPA receptor antagonists, and optionally a benzodiazepine) are prepared as a concentrated aqueous solution. Ordinary metered dose liquid inhalers have poor efficiency for the delivery to the deep lung because the particle size is not sufficiently small (Kim et al., 1985 Am Rev Resp Dis 132:137-142; and Farr et al., 1995 Thorax 50:639-644). These systems are therefore used mostly for local delivery of drugs to the pulmonary airways. In addition, metered doses inhalers may not be able to deliver sufficient volumes of even a concentrated midazolam solution to produce the desired rapid antiseizure effect. Accordingly, in various embodiments, a metered doses inhaler is not used for delivery of the benzodiazepine, e.g., midazolam. In one embodiment a nebulization system with the capability of delivering <5 μm particles (e.g., the PARI LC Star, which has a high efficiency, 78% respirable fraction 0.1-5 μm. see, e.g., pari.com) is used for intrapulmonary administration. Electronic nebulizers which employ a vibrating mesh or aperture plate to generate an aerosol with the required particle size can deliver sufficient quantities rapidly and find use (See, e.g., Knoch and Keller, 2005 Expert Opin Drug Deliv 2: 377-390). Also, custom-designed hand-held, electronic nebulizers can be made and find use.

Aerosolized delivery of the agents (e.g., one or more neurosteroids, one or more AMPA receptor antagonists, and optionally a benzodiazepine) can allow for reduced dosing to achieve desired efficacy, e.g., in comparison to intravenous or intranasal delivery.

In various embodiments, the agents (e.g., one or more neurosteroids, one or more AMPA receptor antagonists, and optionally a benzodiazepine) are dissolved or suspended in an aqueous solution suspended or dissolved in an aqueous solution comprising a glycol and at least one alcohol having five or fewer carbons. In some embodiments, the glycol is selected from the group consisting of ethylene glycol, propylene glycol, and analogs and mixtures thereof. In some embodiments, the alcohol is selected from the group consisting of methanol, ethanol, propanol, isopropanol, and mixtures thereof. In some embodiments, the aqueous solution comprises a glycol:alcohol:water ratio of 7:2:1. In some embodiments, the neurosteroid (e.g., allopregnanolone) is present in a concentration from about 3 mg/mL to about 12 mg/mL, e.g., about 3, 4, 5, 6, 7, 8, 9, 10, 11, 12 mg/mL. In some embodiments, the AMPA receptor antagonist (e.g., perampanel) is present in a concentration from about 1 mg/mL to about 8 mg/mL, e.g., about 1, 2 3, 4, 5, 6, 7, 8 mg/mL.

In various embodiments, the agents (e.g., one or more neurosteroids, one or more AMPA receptor antagonists, and optionally a benzodiazepine) are dissolved or suspended in a cyclodextrin. In varying embodiments, the cyclodextrin is an α-cyclodextrin, a β-cyclodextrin or a γ-cyclodextrin. In varying embodiments, the cyclodextrin is selected from the group consisting of α-cyclodextrin; β-cyclodextrin; γ-cyclodextrin; methyl acyclodextrin; methyl β-cyclodextrin; methyl γ-cyclodextrin; ethyl βcyclodextrin; butyl α-cyclodextrin; butyl β-cyclodextrin; butyl γ-cyclodextrin; pentyl γ-cyclodextrin; hydroxyethyl β-cyclodextrin; hydroxyethyl γcyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl acyclodextrin; acetyl β-cyclodextrin; acetyl γ-cyclodextrin; propionyl βcyclodextrin; butyryl β-cyclodextrin; succinyl α-cyclodextrin; succinyl pcyclodextrin; succinyl γ-cyclodextrin; benzoyl β-cyclodextrin; palmityl pcyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl βcyclodextrin; phosphate ester α-cyclodextrin; phosphate ester β-cyclodextrin; phosphate ester γ-cyclodextrin; 3-trimethylammonium-2-hydroxypropyl βcyclodextrin; sulfobutyl ether β-cyclodextrin; carboxymethyl α-cyclodextrin; carboxymethyl β-cyclodextrin; carboxymethyl γ-cyclodextrin, alkyl cyclodextrins, hydroxy alkyl cyclodextrins, carboxy alkyl cyclodextrins and sulfoalkyl ether cyclodextrins, and combinations thereof. In some embodiments, the neurosteroid, the is dissolved or suspended in an aqueous solution comprising sulfobutyl ether β-cyclodextrin (SBECD). SBECD can include cyclodextrins sold under the trade name DEXOLVE™ and CAPTISOL®. Such formulations are useful for parenteral, e.g., intramuscular, intravenous and/or subcutaneous administration. In some embodiments, the cyclodextrin formulation is a buffered solution having a pH in a relatively neutral pH range, for example, a pH in the range of about 4 to 8, for example, a pH in the range of about 5 to 7. In some embodiments, the one or more active agents (e.g., one or more neurosteroids, one or more AMPA receptor antagonists, and optionally a benzodiazepine) are formulated in a buffered solution, for example, phosphate-buffered saline or a citrate buffer.

In some embodiments, the neurosteroid, e.g., allopregnanolone, and cyclodextrin, e.g., a β-cyclodextrin, e.g., a sulfo butyl ether β-cyclodextrin, e.g., CAPTISOL®, complex is formulated for parenteral administration. In an embodiment, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition. In some embodiments, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid at a concentration between 0.25-30 mg/mL, 0.5-30 mg/mL; 1-30 mg/mL; 5-30 mg/mL, 10-30 mg/mL; 15-30 mg/mL, 0.25-20 mg/mL; 0.5-20 mg/mL; 1-20 mg/mL, 0.5-20 mg/mL; 1-20 mg/mL, 5-20 mg/mL, 10-20 mg/mL, 0.25-15 mg/mL, 0.5-15 mg/mL; 0.5-10 mg/mL; 1-15 mg/mL, 1-10 mg/mL; 1-5 mg/mL; 5-15 mg/mL; 5-10 mg/mL; 10-15 mg/mL; 1-10 mg/mL; 2-8 mg/mL; 2-7 mg/mL; 3-5 mg/mL; 5-15 mg/mL; 7-12 mg/mL; 7-10 mg/mL; 8-9 mg/mL; 3-5 mg/mL; or 3-4 mg/mL. In some embodiments, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid at a concentration of 0.25 mg/mL, 0.5 mg/mL; 1.0 mg/mL; 1.5 mg/mL; 2.0 mg/mL; 2.5 mg/mL; 3.0 mg/mL; 3.5 mg/mL; 4.0 mg/mL; 4.5 mg/mL; 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10 mg/mL, 15 mg/mL, 20 mg/mL, 25 mg·mL, or 30 mg/mL. In an embodiment, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid at a concentration of 1.5 mg/mL. In an embodiment, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid at a concentration of 5 mg/mL. In an embodiment, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid at a concentration of 15 mg/mL.

In some embodiments, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid, e.g., allopregnanolone, at a concentration between 0.25-30 mg/mL, 0.5-30 mg/mL; 1-30 mg/mL; 5-30 mg/mL, 10-30 mg/mL; 15-30 mg/mL, 0.25-20 mg/mL; 0.5-20 mg/mL; 1-20 mg/mL, 0.5-20 mg/mL; 1-20 mg/mL, 5-20 mg/mL, 10-20 mg/mL, 0.25-15 mg/mL, 0.5-15 mg/mL; 0.5-10 mg/mL; 1-15 mg/mL, 1-10 mg/mL; 1-5 mg/mL; 5-15 mg/mL; 5-10 mg/mL; 10-15 mg/mL; 1-10 mg/mL; 2-8 mg/mL; 2-7 mg/mL; 3-5 mg/mL; 5-15 mg/mL; 7-12 mg/mL; 7-10 mg/mL; 8-9 mg/mL; 3-5 mg/mL; or 3-4 mg/mL; and the cyclodextrin, e.g., a β-cyclodextrin, e.g., a sulfo butyl ether β-cyclodextrin, e.g., CAPTISOL®, at a concentration between 25-400 mg/mL; 25-300 mg/mL; 25-200 mg/mL; 25-100 mg/mL; 25-50 mg/mL; 50-400 mg/mL; 50-300 mg/mL; 60-400 mg/mL; 60-300 mg/mL; 150-400 mg/mL; 150-300 mg/mL; 200-300 mg/mL; 200-400 mg/mL; 30-100 mg/mL; 300-400 mg/mL; 30-100 mg/mL; 45-75 mg/mL; 50-70 mg/mL; 55-65 mg/mL; or 50-60 mg/mL. In some embodiments, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid, e.g., allopregnanolone, at a concentration between 0.25-30 mg/mL, 0.5-30 mg/mL; 1-30 mg/mL; 5-30 mg/mL, 10-30 mg/mL; 15-30 mg/mL, 0.25-20 mg/mL; 0.5-20 mg/mL; 1-20 mg/mL, 0.5-20 mg/mL; 1-20 mg/mL, 5-20 mg/mL, 10-20 mg/mL, 0.25-15 mg/mL, 0.5-15 mg/mL; 0.5-10 mg/mL; 1-15 mg/mL, 1-10 mg/mL; 1-5 mg/mL; 5-15 mg/mL; 5-10 mg/mL; 10-15 mg/mL; 1-10 mg/mL; 2-8 mg/mL; 2-7 mg/mL; 3-5 mg/mL; 5-15 mg/mL; 7-12 mg/mL; 7-10 mg/mL; 8-9 mg/mL; 3-5 mg/mL; or 3-4 mg/mL; and the cyclodextrin, e.g., a β-cyclodextrin, e.g., a sulfo butyl ether β-cyclodextrin, e.g., CAPTISOL®, at a concentration of 25 mg/mL; 30 mg/mL; 35 mg/mL; 40 mg/mL; 45 mg/mL; 50 mg/mL; 55 mg/mL; 60 mg/mL; 65 mg/mL; 70 mg/mL; 75 mg/mL; 80 mg/mL; 85 mg/mL; 90 mg/mL, 95 mg/mL; 100 mg/mL; 150 mg/mL; 200 mg/mL; 250 mg/mL; 300 mg/mL; 350 mg/mL; or 400 mg/mL.

In some embodiments, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid, e.g., allopregnanolone, at a concentration between 0.25-30 mg/mL, 0.5-30 mg/mL; 1-30 mg/mL; 5-30 mg/mL, 10-30 mg/mL; 15-30 mg/mL, 0.25-20 mg/mL; 0.5-20 mg/mL; 1-20 mg/mL, 0.5-20 mg/mL; 1-20 mg/mL, 5-20 mg/mL, 10-20 mg/mL, 0.25-15 mg/mL, 0.5-15 mg/mL; 0.5-10 mg/mL; 1-15 mg/mL, 1-10 mg/mL; 1-5 mg/mL; 5-15 mg/mL; 5-10 mg/mL; 10-15 mg/mL; 1-10 mg/mL; 2-8 mg/mL; 2-7 mg/mL; 3-5 mg/mL; 5-15 mg/mL; 7-12 mg/mL; 7-10 mg/mL; 8-9 mg/mL; 3-5 mg/mL; or 3-4 mg/mL; and between 2.5-40%, 2.5-30%, 2.5-20%, 2.5-10%, 5-40%, 5-30%, 5-20%, 5-10%, 6-40%, 6-30%, 6-20%, 6-10%, 10-40%, 10-30%, 10-20%, 20-40%, 20-30%, 25-40%, 25-30%, 3-10%, 4.5-7.5%, 5-7%, 5.5-6.5% of the cyclodextrin, e.g., CAPTISOL®. In some embodiments, the neuroactive steroid, e.g., allopregnanolone, and cyclodextrin, e.g., a β-cyclodextrin, e.g., a sulfo butyl ether β-cyclodextrin, e.g., CAPTISOL®, complex is formulated as an aqueous composition comprising the neuroactive steroid, e.g., allopregnanolone, at a concentration between 0.25-30 mg/mL, 0.5-30 mg/mL; 1-30 mg/mL; 5-30 mg/mL, 10-30 mg/mL; 15-30 mg/mL, 0.25-20 mg/mL; 0.5-20 mg/mL; 1-20 mg/mL, 0.5-20 mg/mL; 1-20 mg/mL, 5-20 mg/mL, 10-20 mg/mL, 0.25-15 mg/mL, 0.5-15 mg/mL; 0.5-10 mg/mL; 1-15 mg/mL, 1-10 mg/mL; 1-5 mg/mL; 5-15 mg/mL; 5-10 mg/mL; 10-15 mg/mL; 1-10 mg/mL; 2-8 mg/mL; 2-7 mg/mL; 3-5 mg/mL; 5-15 mg/mL; 7-12 mg/mL; 7-10 mg/mL; 8-9 mg/mL; 3-5 mg/mL; or 3-4 mg/mL; and 2.5%, 3%, 4%, 4.5%, 5%, 5.5%, 6%, 6.5%, 7%, 7.5%, 8%, 8.5%, 9%, 9.5%, 10%, 15%, 20%, 25%, 30%, 35% or 40% of the cyclodextrin, e.g., CAPTISOL®.

In various embodiments, the agents (e.g., one or more neurosteroids, one or more AMPA receptor antagonists, and optionally a benzodiazepine) are dissolved or suspended in an oil that is edible and/or digestible by the subject, e.g., without undesirable side effects.

In various embodiments, the edible oil comprises one or more vegetable oils. In various embodiments, the vegetable oil is selected from the group consisting of coconut oil, corn oil, cottonseed oil, olive oil, palm oil, peanut oil, rapeseed oil, canola oil, safflower oil, sesame oil, soybean oil, sunflower oil, and mixtures thereof.

In some embodiments, the edible oil comprises one or more nut oils. In some embodiments, the nut oil is selected from the group consisting of almond oil, cashew oil, hazelnut oil, macadamia oil, mongongo nut oil, pecan oil, pine nut oil, pistachio oil, walnut oil, and mixtures thereof.

In some embodiments, the edible oil does not comprise castor oil. In some embodiments, the edible oil does not comprise peanut oil.

Generally, the oils used in the present compositions are isolated from the source, e.g., plant, and used without including further additives (e.g., surfactants, acids (organic or fatty), alcohols, esters, co-solvents, solubilizers, lipids, polymers, glycols) or processing. In various embodiments, the oil vehicle further comprises a preservative (e.g., vitamin E).

The active agents (e.g., one or more neurosteroids, one or more AMPA receptor antagonists, and optionally a benzodiazepine) compositions can be formulated for oral and/or transmucosal delivery using any method known in the art. In one embodiment, the oil-agents (e.g., one or more neurosteroids, one or more AMPA receptor antagonists, and optionally a benzodiazepine) composition is formulated in a capsule, e.g., for oral delivery.

a. Capsules

The capsule shells can be prepared using one or more film forming polymers. Suitable film forming polymers include natural polymers, such as gelatin, and synthetic film forming polymers, such as modified celluloses. Suitable modified celluloses include, but are not limited to, hydroxypropyl methyl cellulose, methyl cellulose, hydroxypropyl methyl cellulose acetate succinate, hydroxypropyl methyl cellulose phthalate, and cellulose acetate phthalate. Hard or soft capsules can be used to administer the hormone. Hard shell capsules are typically prepared by forming the two capsule halves, filling one of the halves with the fill solution, and then sealing the capsule halves together to form the finished capsule. Soft gelatin capsules are typically prepared using a rotary die encapsulation process as described below.

i. Gelatin Capsules

Gelatin is the product of the partial hydrolysis of collagen. Gelatin is classified as either Type A or Type B gelatin. Type A gelatin is derived from the acid hydrolysis of collagen while Type B gelatin is derived from the alkaline hydrolysis of collagen. Traditionally, bovine bones and skins have been used as raw materials for manufacturing Type A and Type B gelatin while porcine skins have been used extensively for manufacturing Type A gelatin. In general, acid-processed gelatins form stronger gels than lime-processed gelatins of the same average molecular weight. The capsules can be formulated as hard or soft gelatin capsules.

ii. Non-Gelatin Capsules

Capsules can be prepared from non-gelatin materials, such as carrageenan or modified celluloses. Carrageenan is a natural polysaccharide hydrocolloid, which is derived from seaweed. It includes a linear carbohydrate polymer of repeating sugar units, without a significant degree of substitution or branching. Most, if not all, of the galactose units on a carrageenan molecule possess a sulfate ester group. There are three main types of carrageenan: kappa, iota and lambda; although minor forms called mu and nu carrageenan also exist.

iii. Shell Additives

Suitable shell additives include plasticizers, opacifiers, colorants, humectants, preservatives, flavorings, and buffering salts and acids, and combinations thereof.

Plasticizers are chemical agents added to gelatin to make the material softer and more flexible. Suitable plasticizers include, but are not limited to, glycerin, sorbitol solutions which are mixtures of sorbitol and sorbitan, and other polyhydric alcohols such as propylene glycol and maltitol or combinations thereof.

Opacifiers are used to opacify the capsule shell when the encapsulated active agents are light sensitive. Suitable opacifiers include titanium dioxide, zinc oxide, calcium carbonate and combinations thereof.

Colorants can be used for marketing and product identification/differentiation purposes. Suitable colorants include synthetic and natural dyes and combinations thereof.

Humectants can be used to suppress the water activity of the softgel. Suitable humectants include glycerin and sorbitol, which are often components of the plasticizer composition. Due to the low water activity of dried, properly stored softgels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl esters (collectively known as “parabens”) or combinations thereof.

Flavorings can be used to mask unpleasant odors and tastes of fill formulations. Suitable flavorings include synthetic and natural flavorings. The use of flavorings can be problematic due to the presence of aldehydes which can cross-link gelatin. As a result, buffering salts and acids can be used in conjunction with flavorings that contain aldehydes in order to inhibit cross-linking of the gelatin.

b. Enteric Capsules

Alternatively, the liquid fills can be incorporated into an enteric capsule, wherein the enteric polymer is a component of the capsule shell, as described in WO 2004/030658 to Banner Pharmacaps, Inc. The enteric capsule shell is prepared from a mass comprising a film-forming polymer, an acid-insoluble polymer which is present in an amount making the capsule resistant to the acid within the stomach, an aqueous solvent, and optionally, one or more plasticizers and/or colorants. Other suitable shell additives including opacifiers, colorants, humectants, preservatives, flavorings, and buffering salts and acids may be added.

i. Film-Forming Polymers

Exemplary film-forming polymers can be of natural or synthetic origin. Natural film-forming polymers include gelatin and gelatin-like polymers. Other suitable natural film-forming polymers include shellac, alginates, pectin, and zeins. Synthetic film-forming polymers include hydroxypropyl methyl cellulose, methyl cellulose, hydroxypropyl methyl cellulose acetate succinate, hydroxypropyl methyl cellulose phthalate, cellulose acetate phthalate, and acrylates such as poly (meth)acrylate. The weight ratio of acid-insoluble polymer to film-forming polymer is from about 15% to about 50%. In one embodiment, the film forming polymer is gelatin.

ii. Acid-Insoluble Polymers

Exemplary acid-insoluble polymers include cellulose acetate phthalate, cellulose acetate butyrate, hydroxypropyl methyl cellulose phthalate, algenic acid salts such as sodium or potassium alginate, shellac, pectin, acrylic acid-methylacrylic acid copolymers (available under the tradename EUDRAGIT® from Rohm America Inc., Piscataway, N.J. as a powder or a 30% aqueous dispersion; or under the tradename EASTACRYL®, from Eastman Chemical Co., Kingsport, Tenn., as a 30% dispersion). In one embodiment, the acid-insoluble polymer is EUDRAGIT® L100, which is a methacrylic acid/methacrylic acid methyl ester copolymer. The acid-insoluble polymer is present in an amount from about 8% to about 20% by weight of the wet gelatin mass. The weight ratio of acid-insoluble polymer to film-forming polymer is from about 15% to about 50%.

iii. Aqueous Solvent

Hard and soft capsules are typically prepared from solutions or suspensions of the film forming polymer and the acid-insoluble polymer. Suitable solvents include water, aqueous solvents, and organic solvents. In one embodiment, the solvent is water or an aqueous solvent. Exemplary aqueous solvents include water or aqueous solutions of alkalis such as ammonia, sodium hydroxide, potassium hydroxide, ethylene diamine, hydroxylamine, tri-ethanol amine, or hydroalcoholic solutions of the same. The alkali can be adjusted such that the final pH of the gelatin mass is less than or equal to 9.0, preferably less than or equal to 8.5, more preferably less than or equal to 8.0. In one embodiment, the alkali is a volatile alkali such as ammonia or ethylene diamine. Upon drying of the finished capsule, the water content of the capsule is from about 2% to about 10% by weight of the capsule, preferably from about 4% to about 8% by weight of the capsule.

iv. Plasticizers

Exemplary plasticizers include glycerol, glycerin, sorbitol, polyethylene glycol, citric acid, citric acid esters such as triethylcitrate, polyalcohols with 3-6 carbons and combinations thereof. The plasticizer to polymer (film forming polymer plus acid-insoluble polymer) ratio is from about 10% to about 50% of the polymer weight.

c. Methods of Manufacture

i. Capsule Fill

The fill material is prepared by dissolving the neurosteroid (e.g., allopregnanolone) in the carrier containing a fatty acid solvent, such as oleic acid. The mixture of hormone and fatty acid may be heated to facilitate dissolution of the hormone. Upon cooling to room temperature and encapsulation, the solution remains a liquid. The fill is typically deaerated prior to encapsulation in a soft gelatin capsule. Additional excipients including, but not limited to, co-solvents, antioxidants may be added to the mixture of the hormone and fatty acid. Again the mixture may be heated to facilitate dissolution of the excipients. The neurosteroid (e.g., allopregnanolone) is fully dissolved in the carrier and remains so upon storage.

ii. Capsule Shell

a. Gelatin or Non-Gelatin Capsules

The main ingredients of the capsule shell are gelatin (or a gelatin substitute for non-gelatin capsules), plasticizer, and purified water. The primary difference between soft and hard capsules is the amount of plasticizer present in the capsule shell.

Typical gel formulations contain (w/w) 40-50% gelatin, 20-30% plasticizer, and 30-40% purified water. Most of the water is subsequently lost during capsule drying. The ingredients are combined to form a molten gelatin mass using either a cold melt or a hot melt process. The prepared gel masses are transferred to preheated, temperature-controlled, jacketed holding tanks where the gel mass is aged at 50-60° C. until used for encapsulation.

i. Cold Melt Process

The cold melt process involves mixing gelatin with plasticizer and chilled water and then transferring the mixture to a jacket-heated tank. Typically, gelatin is added to the plasticizer at ambient temperature (18-22° C.). The mixture is cooked (57-95° C.) under vacuum for 15-30 minutes to a homogeneous, deaerated gel mass. Additional shell additives can be added to the gel mass at any point during the gel manufacturing process or they may be incorporated into the finished gel mass using a high torque mixer.

ii. Hot Melt Process

The hot melt process involves adding, under mild agitation, the gelatin to a preheated (60-80° C.) mixture of plasticizer and water and stirring the blend until complete melting is achieved. While the hot melt process is faster than the cold melt process, it is less accurately controlled and more susceptible to foaming and dusting.

b. Soft Capsules

Soft capsules are typically produced using a rotary die encapsulation process. The gel mass is fed either by gravity or through positive displacement pumping to two heated (48-65° C.) metering devices. The metering devices control the flow of gel into cooled (10-18° C.), rotating casting drums. Ribbons are formed as the cast gel masses set on contact with the surface of the drums.

The ribbons are fed through a series of guide rolls and between injection wedges and the capsule-forming dies. A food-grade lubricant oil is applied onto the ribbons to reduce their tackiness and facilitate their transfer. Suitable lubricants include mineral oil, medium chain triglycerides, and soybean oil. Fill formulations are fed into the encapsulation machine by gravity. In the preferred embodiment, the soft capsules contain printing on the surface, optionally identifying the encapsulated agent and/or dosage.

Upon drying of the finished capsule, the water content of the capsule is from about 2% to about 10% by weight of the capsule, preferably from about 4% to about 8% by weight of the capsule.

c. Enteric Capsules

A method of making an enteric capsule shell is described in WO 2004/030658 to Banner Pharmacaps, Inc. The enteric mass is typically manufactured by preparing an aqueous solution comprising a film-forming, water soluble polymer and an acid-insoluble polymer and mixing the solution with one or more appropriate plasticizers to form a gelatin mass. Alternatively, the enteric mass can be prepared by using a ready-made aqueous dispersion of the acid-insoluble polymer by adding alkaline materials such as ammonium, sodium, or potassium hydroxides or other alkalis that will cause the acid-insoluble polymer to dissolve. The plasticizer-wetted, film-forming polymer can then be mixed with the solution of the acid-insoluble polymer. The mass can also be prepared by dissolving the acid-insoluble polymer or polymers in the form of salts of the above-mentioned bases or alkalis directly in water and mixing the solution with the plasticizer-wetted, film-forming polymer. The mass is cast into films or ribbons using heat controlled drums or surfaces. The fill material is encapsulated in a soft capsule using a rotary die. The capsules are dried under controlled conditions of temperature and humidity. The final moisture content of the shell composition is from about 2% to about 10% by weight of the capsule shell, preferably from about 4% to about 8% by weight by weight of the capsule shell.

Alternatively, release of the agents (e.g., the one or more neurosteroids, one or more AMPA receptor antagonists, and optionally a benzodiazepine) from the capsule can be modified by coating the capsule with one or more modified release coatings, such as sustained release coatings, delayed release coatings, and combinations thereof.

### Dosing

Appropriate dosing will depend on the size and health of the patient and can be readily determined by a trained clinician. Initial doses are low and then can be incrementally increased until the desired therapeutic effect is achieved with little or no adverse side effects. Determination of an effective amount for administration in a single dosage is well within the capability of those skilled in the art, especially in light of the detailed disclosure provided herein. Generally, an efficacious or effective amount of the agents (e.g., the one or more neurosteroids, one or more AMPA receptor antagonists, and optionally a benzodiazepine) is determined by first administering a low dose or small amount of the agent and then incrementally increasing the administered dose or dosages, adding a second or third medication as needed, until a desired effect of is observed in the treated subject with minimal or no toxic side effects. Applicable methods for determining an appropriate dose and dosing schedule for administration of a combination of agents are described, for example, in Goodman & Gilman's The Pharmacological Basis of Therapeutics, 13th edition, 2017, McGraw-Hill, supra; in a Physicians' Desk Reference (PDR), PDR Network; 71st 2017 ed. edition (Dec. 13, 2016); in Remington: The Science and Practice of Pharmacy, 21^{st} Ed., 2005, supra; and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, each of which are hereby incorporated herein by reference.

The concentration of the agents (e.g., the one or more neurosteroids, one or more AMPA receptor antagonists, and optionally a benzodiazepine) in the vehicle (e.g., cyclodextrin and/or edible oil) is preferably in unit dosage form. The term “unit dosage form”, as used in the specification, refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the novel unit dosage forms are governed by and directly dependent on (a) the unique characteristics of the active material and the particular effect to be achieved and (b) the limitations inherent in the art of compounding such an active material for use in humans and animals.

In various embodiments, the neurosteroid is administered at a dose that is less than about 10%, 15%, 25%, 50% or 75% of established doses for their administration for the prevention or mitigation of an epileptic seizure, or for mitigation of depression symptoms. In some embodiments, the neurosteroid is administered at a dose in the range of about 0.5 mg/kg to about 10.0 mg/kg, for example, about 0.5 mg/kg to about 1 mg/kg, 2 mg/kg, 3 mg/kg, 4 mg/kg, 5 mg/kg, 6 mg/kg, 7 mg/kg, 8 mg/kg, 9 mg/kg, 10.0 mg/kg, 20 mg/kg or 30 mg/kg. When co-administered with one or more AMPA receptor antagonists, and optionally a benzodiazepine, the neurosteroid can be co-administered at a dose that is less than about 10%, 15%, 25%, 50% or 75% of the aforementioned doses or at a dose that is less than about 10%, 15%, 25%, 50% or 75% of established doses for their administration for the prevention or mitigation of an epileptic seizure, or for mitigation of depression symptoms. When co-administered with one or more AMPA receptor antagonists, and optionally a benzodiazepine, the neurosteroid can be co-administered at a dose that is less than about 10%, 15%, 25%, 50% or 75% of doses known to be efficacious via a selected route of administration (e.g., oral, intramuscular, intravenous, subcutaneous and/or intrapulmonary).

In various embodiments, the AMPA receptor antagonist is administered at a dose that is less than about 10%, 15%, 25%, 50% or 75% of established doses for their administration for the prevention or mitigation of an epileptic seizure, or for mitigation of depression symptoms. In some embodiments, the AMPA receptor antagonist is administered at a dose in the range of about 0.5 mg/kg to about 4.0 mg/kg, for example, about 0.5 mg/kg to about 1 mg/kg, 1.5 mg/kg, 2.0 mg/kg, 2.5 mg/kg, 3.0 mg/kg, 3.5 mg/kg, or 4.0 mg/kg. When co-administered with one or more neurosteroids and optionally, a benzodiazepine, the AMPA receptor antagonist can be co-administered at a dose that is less than about 10%, 15%, 25%, 50% or 75% of the aforementioned doses or at a dose that is less than about 10%, 15%, 25%, 50% or 75% of established doses for their administration for the prevention or mitigation of an epileptic seizure, or for mitigation of depression symptoms. When co-administered with one or more neurosteroids and optionally, a benzodiazepine, the AMPA receptor antagonist can be co-administered at a dose that is less than about 10%, 15%, 25%, 50% or 75% of doses known to be efficacious via a selected route of administration (e.g., oral, intramuscular, intravenous, subcutaneous and/or intrapulmonary).

In various embodiments, the benzodiazepines are administered at a dose that is less than about 10%, 15%, 25%, 50% or 75% of established doses for their administration for the prevention or mitigation of an epileptic seizure, or for mitigation of depression or anxiety symptoms. In some embodiments, the benzodiazepine is administered at a dose in the range of about 0.5 mg/kg to about 4.0 mg/kg, for example, about 0.5 mg/kg to about 1 mg/kg, 1.5 mg/kg, 1.8 mg/kg, 2.0 mg/kg, 2.5 mg/kg, 3.0 mg/kg, 3.5 mg/kg, or 4.0 mg/kg. In some embodiments the benzodiazepine is administered at a dose in the range of about 10 μg/kg to about 80 μg/kg, for example, about 20 μg/kg to about 60 μg/kg, for example, about 25 μg/kg to about 50 μg/kg, for example, about 10 μg/kg, 15 μg/kg, 20 μg/kg, 25 μg/kg, 30 μg/kg, 35 μg/kg, 40 μg/kg, 45 μg/kg, 50 μg/kg, 60 μg/kg, 70 μg/kg, or 80 μg/kg. In some embodiments, the benzodiazepine is administered at a dose in the range of about 0.3 μg/kg to about 3.0 μg/kg. In varying embodiments, the benzodiazepine is administered at a dose that does not decrease blood pressure. When co-administered with one or more neurosteroids and one or more AMPA receptor antagonists, the benzodiazepine can be co-administered at a dose that is less than about 10%, 15%, 25%, 50% or 75% of the aforementioned doses or at a dose that is less than about 10%, 15%, 25%, 50% or 75% of established doses for their administration for the prevention or mitigation of an epileptic seizure, or for mitigation of depression or anxiety symptoms. When co-administered with one or more neurosteroids and one or more AMPA receptor antagonists, the benzodiazepine can be co-administered at a dose that is less than about 10%, 15%, 25%, 50% or 75% of doses known to be efficacious via a selected route of administration (e.g., oral, intramuscular, intravenous, subcutaneous and/or intrapulmonary).

In various embodiments, the compositions are formulated for administration of a benzodiazepine, a neurosteroid and an AMPA receptor antagonist, each at a dose in the range of about 0.5 mg/kg to about 50 mg/kg, e.g., about 0.5 mg/kg, 1.0 mg/kg, 1.5 mg/kg, 1.8 mg/kg, 2.0 mg/kg, 2.5 mg/kg, 3 mg/kg, 4 mg/kg, 5 mg/kg, 6 mg/kg, 10 mg/kg, 15 mg/kg, 20 mg/kg, 25 mg/kg, 30 mg/kg, 35 mg/kg, 40 mg/kg, 45 mg/kg, or 50 mg/kg. When co-administered with one or more AMPA receptor antagonists, and optionally a benzodiazepine, the neurosteroid (e.g., allopregnanolone) can be co-administered at a dose that is less than about 10%, 15%, 25%, 50% or 75% of the aforementioned doses or at a dose that is less than about 10%, 15%, 25%, 50% or 75% of established doses for their administration for the prevention or mitigation of an epileptic seizure. When co-administered with one or more AMPA receptor antagonists and optionally a benzodiazepine, the neurosteroid can be co-administered at a dose that is less than about 10%, 15%, 25%, 50% or 75% of doses known to be efficacious via a selected route of administration (e.g., oral, intramuscular, intravenous, subcutaneous and/or intrapulmonary).

In an embodiment, the method includes acute treatment of a disorder described herein. In such cases, the patient receives treatment as soon as possible, e.g., within 6, 5, 4, 3, 2, 1 hours or less, after exposure to a nerve agent or after experiencing a seizure. For example, in an embodiment, a method described herein provides relief from a symptom described herein in less than 1 week (e.g., within 6 days, 5 days, 4 days, 3 days, 2 days, 1 day, 12 hours, 8 hours, 4 hours, 2 hours or 1 hour). In an embodiment, the subject experiences, upon administration of the combined active agents described herein (e.g., a neurosteroid, an AMPA receptor antagonist, and optionally a benzodiazepine) rapid onset of efficacy of the combined active agents. For example, in an embodiment, a subject experiences relief from a symptom of a disorder described herein within 1 week (e.g., within 6 days, 5 days, 4 days, 3 days, 2 days, 1 day, 12 hours, 8 hours, 4 hours, 2 hours or 1 hour).

In an embodiment, a methods provide for sustained efficacy upon treatment with the combined active agents described herein (e.g., a neurosteroid, an AMPA receptor antagonist, and optionally a benzodiazepine). For example, in an embodiment, a subject is treated with the combined active agents described herein, wherein the treatment effectively treats a symptom of a disorder described herein and the efficacy is maintained for at least 1 day (e.g., at least 2 days, 3 days, 4 days, 5 days, 6 days, 1 week, 2 weeks, 3 weeks, 1 month, 2 months, 3 months, 4 months, 5 months, or 6 months). In an embodiment, the efficacy is maintained after a single course of treatment of the combined active agents described herein (e.g., a neurosteroid, an AMPA receptor antagonist, and optionally a benzodiazepine). Course of treatment, as described herein is a treatment regimen administered to a subject so as to provide efficacy of a symptom of a disorder to the subject. In an embodiment, a course of treatment is a single dose. In another embodiment, a course of treatment includes multiple doses of the combined active agents described herein. In another embodiment, a course of treatment includes a cycle of treatment of the combined active agents described herein.

In an embodiment, a method described herein can include a course of treatment with multiple dosages or cycles of treatment, for example, where a first dose or cycle of treatment is a parenteral dose such as an i.v. dose, and a second dose or cycle of treatment is an oral dose.

In some embodiments, the administering comprises administering one or more cycles of treatment, wherein a cycle of treatment comprises: administering a first dose of the combined active agents, administering a second dose of the combined active agents, and administering a third dose of the combined active agents, the doses being sufficient to treat said subject.

In some embodiments, a cycle of treatment comprises a first (titration, ramp-up, step-up, loading) dose, a second (maintenance) dose, and a third (taper, weaning, step-down) dose. In some embodiments, the administering comprises administering one or more cycles of treatment, wherein a cycle of treatment comprises: administering a first dose of the neuroactive steroid, administering a second dose of the neuroactive steroid, and administering a third dose of the neuroactive steroid, said neuroactive steroid doses being sufficient to treat said subject.

In some embodiments, the administering (or course of administration) comprises administering more than one cycle of treatment (e.g., two cycles of treatment, three cycles of treatment). In some embodiments, a rest period follows (e.g., immediately follows, is less than 60, 30, 20, 10, 5, 2, or 1 minute after) the first cycle of treatment. In some embodiments, a rest period precedes the second cycle of treatment. In some embodiments, a rest period follows the first cycle of treatment and precedes the second cycle of treatment. In some embodiments, the rest period is 6 to 8 days (e.g., 7 days) in duration.

In some embodiments, [0028] In some embodiments, the first (titration) dose comprises administering a plurality of step doses (e.g., a first, second, and third step dose). In some embodiments, the first step dose is 20 to 40 μg/kg/hr (e.g., about 30 μg/kg/hr, 29 rig/kg/hr). In some embodiments, the second step dose is 45 to 65 rig/kg/hr (e.g., about 60 μg/kg/hr, 58 μg/kg/hr). In some embodiments, the third step dose is 80 to 100 μg/kg/hr (e.g., about 90 μg/kg/hr, 86 μg/kg/hr). In some embodiments, each of the first, second, and third step doses are 2 to 6 hours (e.g., 4 hours) in duration. In some embodiments, each of the first, second, and third step doses are 1, 2, 3, 4, 5, or 6 hours in duration. In some embodiments, each of the first, second, and third step doses are administered for equal periods of duration.

In an embodiment, the first (titration) dose is followed by a second (maintenance) dose. In an embodiment, the second (maintenance) dose is administered within 2 hours after the first (titration) dose, e.g., within 1 hour, 30 minutes, 15 minutes, or less. In an embodiment, the second (maintenance) dose or neurosteroid is from about 70 to 175 μg/kg/hr, e.g., from about 90 to about 150 μg/kg/hr. In some embodiments, the second (maintenance) dose comprises administering a single (constant) dose of neuroactive steroid/unit time. In one embodiment, treatment involves administering a first/step-up infusion dose at an amount of neurosteroid/unit time of 5-100 μg/kg/hour, 10-80 μg/kg/hour, or 15-70 μg/kg/hour; administering a second/maintenance infusion of neurosteroid at an amount of neurosteroid/unit time of 50-100 μg/kg/hour, 70-100 μg/kg/hour, or 86 μg/kg/hour; and administering a third infusion of neurosteriod, said third/downward taper infusion comprising administering neurosteroid at an amount of neurosteroid/unit time of 5-100 μg/kg/hour, 10-80 μg/kg/hour, or 15-70 μg/kg/hour.

5. Monitoring Efficacy

Co-administration of one or more neurosteroids, and one or more AMPA receptor antagonists, and optionally a benzodiazepine, to a subject results in the prevention of the occurrence of an impending seizure and/or the rapid termination or abortion of a seizure in progress.

In various embodiments, efficacy can be monitored by the subject. For example, in a subject experiencing aura or receiving a warning from a seizure prediction device, or experiencing depression symptoms, the subject can self-administer the active agents. If the active agents are administered in an efficacious amount, the sensation of aura should subside and/or the seizure prediction device should no longer predict the imminent occurrence of an impending seizure. If the sensation of aura does not subside and/or the seizure prediction device continues to predict an impending seizure, a second dose of active agents can be administered.

In other embodiments, the efficacy is monitored by a caregiver. For example, in a subject experiencing the onset of a seizure or in situations where a seizure has commenced, the subject may require intrapulmonary administration of the active agents by a caregiver. If the active agents are administered in an efficacious amount, the seizure, along with the subject's symptoms of the seizure, should rapidly terminate or abort. If the seizure does not terminate, further doses of the active agents can be administered.

6. Kits

The pharmaceutical compositions and neurosteroid, AMPA receptor antagonist, and optional benzodiazepine, active agent combinations can be provided in a kit. In certain embodiments, a kit comprises one or more neurosteroids one or more AMPA receptor antagonists, and optionally one or more benzodiazepines, in separate formulations. In certain embodiments, the kits comprise one or more neurosteroids, one or more AMPA receptor antagonists, and optionally one or more benzodiazepines, within the same formulation. In varying embodiments, the neurosteroid, the AMPA receptor antagonist, and optionally the benzodiazepine are provided in subtherapeutic or non-therapeutic doses or amounts. In certain embodiments, the kits provide the one or more neurosteroids, one or more AMPA receptor antagonists, and optionally, one or more benzodiazepines, independently in uniform dosage formulations throughout the course of treatment. In certain embodiments, the kits provide the one or more neurosteroids, one or more AMPA receptor antagonists, and optionally one or more benzodiazepines, in graduated dosages over the course of treatment, either increasing or decreasing, but usually increasing to an efficacious dosage level, according to the requirements of an individual.

In some embodiments, the neurosteroid is selected from the group consisting of allopregnanolone, allotetrahydrodeoxycorticosterone, ganaxolone, alphaxolone, alphadolone, hydroxydione, minaxolone, and Althesin. In some embodiments, the neurosteroid is allopregnanolone. In some embodiments, the kit comprises allopregnanolone, perampanel, and optionally, a benzodiazepine selected from the group consisting of midazolam, lorazepam, and diazepam. In some embodiments, the AMPA receptor antagonist is selected from the group consisting of perampanel, selurampanel, talampanel, tezampanel, fanapanel (a.k.a., ZK-200775), irampanel, kynurenic acid, CFM-2, CNQX, CNQX disodium salt, CP 465022 hydrochloride, DNQX, DNQX disodium salt, Evans Blue tetrasodium salt, GYKI 47261 dihydrochloride, GYKI 52466 dihydrochloride, GYKI 53655 hydrochloride, IEM 1925 dihydrobromide, Naspm trihydrochloride, NBQX, NBQX disodium salt, Philanthotoxin 74, SYM 2206, UBP 282, and YM 90K hydrochloride. In some embodiments, the benzodiazepine is selected from the group consisting of bretazenil, clonazepam, cloxazolam, clorazepate, diazepam, fludiazepam, flutoprazepam, lorazepam, midazolam, nimetazepam, nitrazepam, phenazepam, temazepam and clobazam. In some embodiments, the benzodiazepine is selected from the group consisting of midazolam, lorazepam and diazepam.

In some embodiments, one or more the neurosteroid, the AMPA receptor antagonist, and optionally the benzodiazepine, is formulated for inhalational, intranasal or intrapulmonary administration. In some embodiments, one or more of the neurosteroid, the AMPA receptor antagonist, and optionally, the benzodiazepine, is formulated for oral or parenteral delivery. In some embodiments, one or more of the neurosteroid, the AMPA receptor antagonist, and optionally, the benzodiazepine, are formulated for a parenteral route selected from the group consisting of inhalational, intrapulmonary, intranasal, intramuscular, subcutaneous, transmucosal and intravenous. In some embodiments, the benzodiazepine is an agonist of the benzodiazepine recognition site on GABA_{A} receptors and stimulates endogenous neurosteroid synthesis. In some embodiments, the neurosteroid is suspended or dissolved in a cyclodextrin (e.g., α-cyclodextrin; β-cyclodextrin; γ-cyclodextrin; methyl acyclodextrin; methyl β-cyclodextrin; methyl γ-cyclodextrin; ethyl βcyclodextrin; butyl α-cyclodextrin; butyl β-cyclodextrin; butyl γ-cyclodextrin; pentyl γ-cyclodextrin; hydroxyethyl β-cyclodextrin; hydroxyethyl γcyclodextrin; 2-hydroxypropyl ca-cyclodextrin; 2-hydroxypropyl β-cyclodextrin; 2-hydroxypropyl γ-cyclodextrin; 2-hydroxybutyl β-cyclodextrin; acetyl acyclodextrin; acetyl β-cyclodextrin; acetyl γ-cyclodextrin; propionyl pcyclodextrin; butyryl β-cyclodextrin; succinyl α-cyclodextrin; succinyl βcyclodextrin; succinyl γ-cyclodextrin; benzoyl β-cyclodextrin; palmityl βcyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl pcyclodextrin; phosphate ester α-cyclodextrin; phosphate ester β-cyclodextrin; phosphate ester γ-cyclodextrin; β-trimethylammonium-2-hydroxypropyl pcyclodextrin; sulfobutyl ether β-cyclodextrin; carboxymethyl α-cyclodextrin; carboxymethyl β-cyclodextrin; carboxymethyl γ-cyclodextrin, alkyl cyclodextrins, hydroxy alkyl cyclodextrins, carboxy alkyl cyclodextrins and sulfoalkyl ether cyclodextrins, and combinations thereof). In some embodiments, the cyclodextrin is a beta-cyclodextrin disclosed in U.S. Pat. Nos. 5,874,418; 6,046,177; or 7,635,733, which are hereby herein incorporated by reference in their entireties for all purposes. In some embodiments, the neurosteroid, the AMPA receptor antagonist, and optionally, the benzodiazepine, are dissolved or suspended in an aqueous solution comprising sulfobutyl ether β-cyclodextrin (SBECD). SBECD can include cyclodextrins sold under the trade name DEXOLVE™ and CAPTISOL®.

In some embodiments, the neurosteroid is suspended or dissolved in an edible oil. In some embodiments, the edible oil comprises one or more vegetable oils. In some embodiments, the vegetable oil is selected from the group consisting of coconut oil, corn oil, cottonseed oil, olive oil, palm oil, peanut oil, rapeseed oil, canola oil, safflower oil, sesame oil, soybean oil, sunflower oil, and mixtures thereof. In some embodiments, the edible oil is canola oil. In some embodiments, the edible oil comprises one or more nut oils. In some embodiments, the nut oil is selected from the group consisting of almond oil, cashew oil, hazelnut oil, macadamia oil, mongongo nut oil, pecan oil, pine nut oil, pistachio oil, walnut oil, and mixtures thereof.

### EXAMPLES

The following examples are offered to illustrate, but not to limit the claimed invention.

### Example 1

### Combination of Midazolam, Allopregnanolone and Perampanel Protect Rats Against Organophosphate-Induced Status Epilepticus and Associated EEG Changes

In the present example, we explored whether the combination of midazolam (a benzodiazepine) and allopregnanolone (a neurosteroid), also known as dual therapy, is protective against DFP (diisopropylfluorophosphate)-induced status epilepticus in rats. Further, we studied the effect of addition of perampanel (an AMPA ionotropic receptor antagonist) to midazolam and allopregnanolone, also known as triple therapy, in this animal model.

### Methods

Animals.

Adult male Sprague-Dawley rats (SD; Charles-Rivers, 250-400 g) were used in the present study.

Status epilepticus was induced in SD rats with a subcutaneous injection of DFP (4 mg/kg). The rats were administered with atropine sulfate (2 mg/kg, IM) and pralidoxime (2-PAM; 25 mg/kg, IM) 1 min after DFP administration.

Continuous video EEG was monitored from permanently implanted cortical electrodes before and for at least 5 h after DFP exposure.

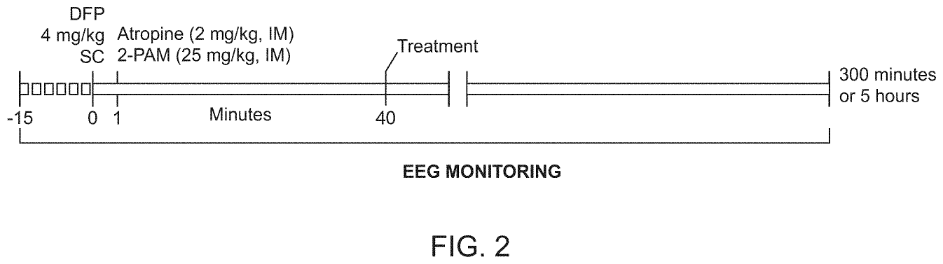

Midazolam (1.8 mg/kg, IM) or dual therapy (Midazolam 1.8 mg/kg, IM+Allopregnanolone 6 mg/kg, IM) or triple therapy (Midazolam 1.8 mg/kg, IM+Allopregnanolone 6 mg/kg, IM+Perampanel 2 mg/kg, IM) or vehicle control (IM, vehicle midazolam+vehicle allopregnanolone+vehicle perampanel) were administered 40 min after DFP administration as shown in FIG. 2.

### Results

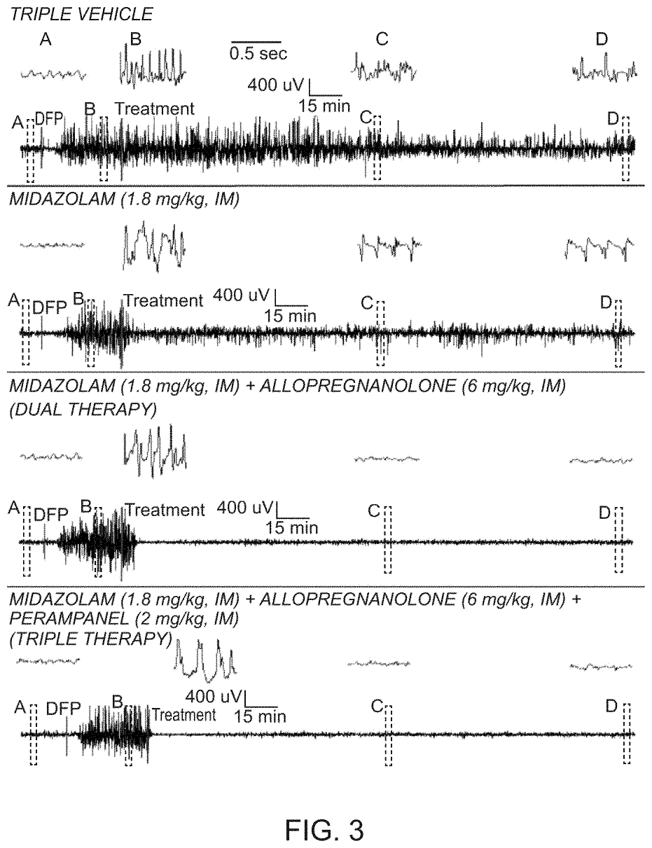

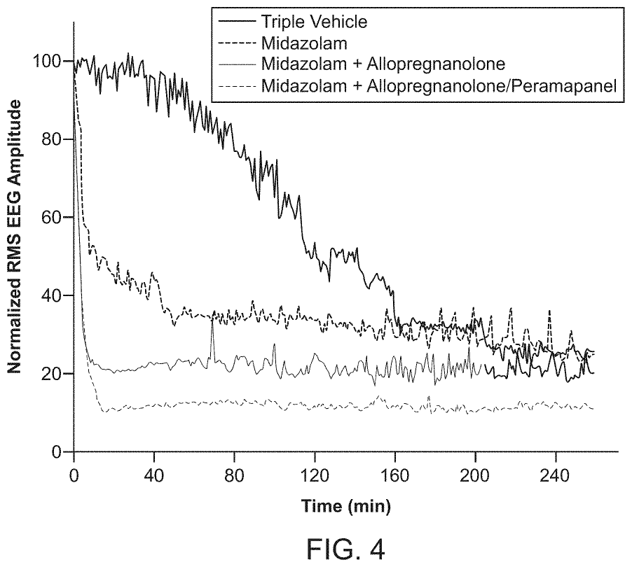

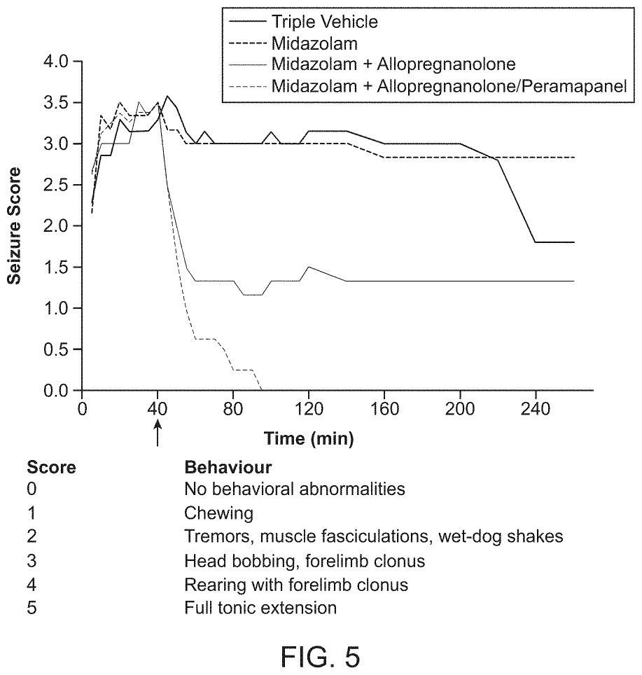

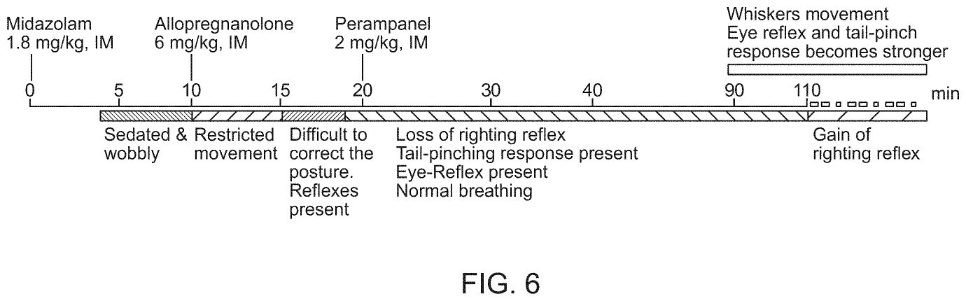

Results of representative EEGs are provided in FIG. 3. Results of root mean square EEG amplitude are provided in FIG. 4. Scoring of behavioral observations is provided in FIG. 5. Results of observations of the righting reflex are provided in FIG. 6.

### Discussion

DFP resulted in robust status epilepticus within few minutes of its injection in SD rats. Midazolam (1.8 mg/kg, IM) alone was in-effective in terminating status epilepticus. Dual combination of midazolam (1.8 mg/kg, IM) and allopregnanolone (6 mg/kg, IM) rapidly terminated status epilepticus and normalized RMS EEG amplitude in 83.33% of the animals tested. Triple combination of midazolam (1.8 mg/kg, IM) and allopregnanolone (6 mg/kg, IM) and perampanel (2 mg/kg, IM) stopped behavioral and electrical seizures in 100% of the animals tested; the effect could be seen within few minutes of its administration. There was no mortality in animals treated with midazolam alone, dual or triple combinations. However, DFP treated animals injected 40 min later with triple vehicle resulted in 33.33% mortality. Both dual or triple therapy resulted in sedation in these animals; however the animals were found to behave completely normal after the sedation is over. In conclusion, both dual or triple therapy hold promise in the management of DFP-induced status epilepticus.

### Example 2

### A Single Intramuscular Injection of Allopregnanolone and Perampanel on Top of Standard-of-Care Midazolam Protect Rats Against Organophosphate-Induced Status Epilepticus and Associated EEG Changes

### Methods

Animals.

Male SD rats (250-450 g; Charles River Laboratories) were kept in a vivarium under controlled environmental conditions (temperature, 22-26° C.; relative humidity, 40-50%) with an artificial 12-h light/dark cycle. Experiments were performed during the light phase of the light/dark cycle after a minimum 30 min period of acclimation to the experimental room. The animal facilities were fully accredited by the Association for Assessment and Accreditation of Laboratory Animal Care. All studies were performed under protocols approved by the Animal Care and Use Committee of the University of California, Davis in strict compliance with the Guide for the Care and Use of Laboratory Animals of the National Research Council (National Academy Press, Washington, D.C.). All animals injected with DFP (Diisopropyl fluorophosphates; an organophosphate) were administered 5% Dextrose (s.c.) 5 h after its challenge and returned to their respective cages. These animals were put back to the vivarium at the end of their recordings. Animals were given soft chow until they were fitted to regain normal consumption of water and solid food.

Implantation of Cortical Electrodes.

Rats were implanted with Electroencephalographic (EEG) electrodes in order to monitor the electric activity in the brain following DFP intoxication. Animals were anesthetized using ketamine (60 mg/kg., i.p.) and dexmedetomidine (0.5 mg/kg., i.p) and were stabilized in a stereotaxic apparatus. Six recording screws were implanted epidurally, three on each side of the sagittal skull suture. A 6-pin rat implant (Pinnacle 8239SE3) was connected to the screws. The head-mount was fixed using dental acrylic cement. Ketoprofen (5 mg/kg, SC), an analgesic, was administered during the surgery and the following day. At least 7-10 days were allowed for the recovery from the surgical procedure.

DFP Treatment Paradigm.

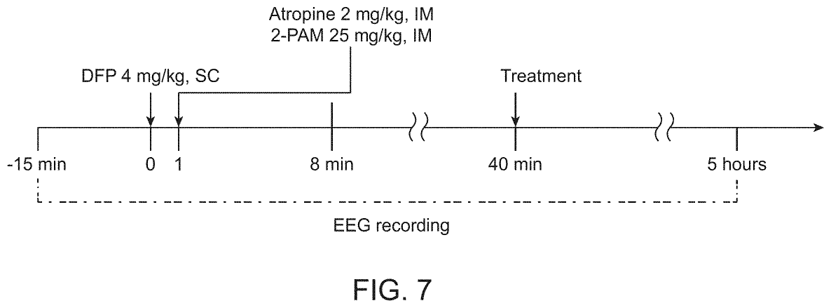

The treatment schedule is illustrated in FIG. 7. Rats were randomly divided into different treatment groups. All animals received DFP injection (4 mg/kg) subcutaneously in an injection volume of 300 μl prepared in 0.1 M sodium phosphate buffer (pH=7.2). Atropine sulfate (2 mg/kg, IM) and 2-PAM (2-pyridine aldoxime methyl chloride; 25 mg/kg, IM) were administered as two separate injections 1 minute after DFP injection. Animals were observed for at least 5 h after DFP injection. One can observe continuous seizure activity (both behaviorally and electrographically) within 5-10 min of DFP administration. After 40 min of DFP injection (when the animals are supposed to be refractory to anti-seizure medications), animals received different treatment options. All IACUC endpoints were followed in administering these molecules.

EEG Measurements.

Behavioral and electrographic seizures were monitored using Pinnacle 8401 data conditioning & Acquisition system (Pinnacle Technology, Lawrence, Kans.). Rats were allowed to move freely in the monitoring system during the recordings. EEG recordings were reviewed using the Sirenia Seizure Pro software (Pinnacle Technology, Lawrence, Kans.) by expert researchers. The recordings were analyzed using a real-time and offline signal analysis using Sigview spectrum analyzer software, ver. 3.0.2. The power of EEG spectrum was calculated as the root mean square value (RMS EEG amplitude) of each 1 min epoch during a 5 h recording. The obtained RMS value through SigView was corrected by calculating the percentage reduction in the RMS values with respect to its baseline (Normalized EEG RMS amplitude) of the individual animal. The results were compared between control and treatment groups.

### Results

Preparation of Allopregnanolone (a Neurosteroid) and Perampanel (an AMPA Receptor Antagonist) Solutions

Two scenarios were used to prepare allopregnanolone or perampanel solutions for testing their efficacy in DFP-induce status epilepticus animal model.

In the first scenario, we have separately prepared allopregnanolone (6 mg/ml) and perampanel (4 mg/ml) solutions. After 40 min of DFP challenge, rats received standard-of-care midazolam (1.8 mg/kg, IM) followed immediately by two separate intramuscular injections of allopregnanolone (6 mg/kg) and perampanel (2 mg/kg) in the hind thighs. The individual solutions were prepared as follows:

(A) Allopregnanolone (6 mg/ml) was prepared in 24% w/v β-cyclodextrin in saline using sonication technique.

(B) Perampanel (4 mg/ml) was prepared in using propylene glycol:ethanol:water in the ratio of 7:2:1. The solution was prepared with intermittent sonication and warming

(C) A commercial formulation of midazolam (5 mg/ml) was used.

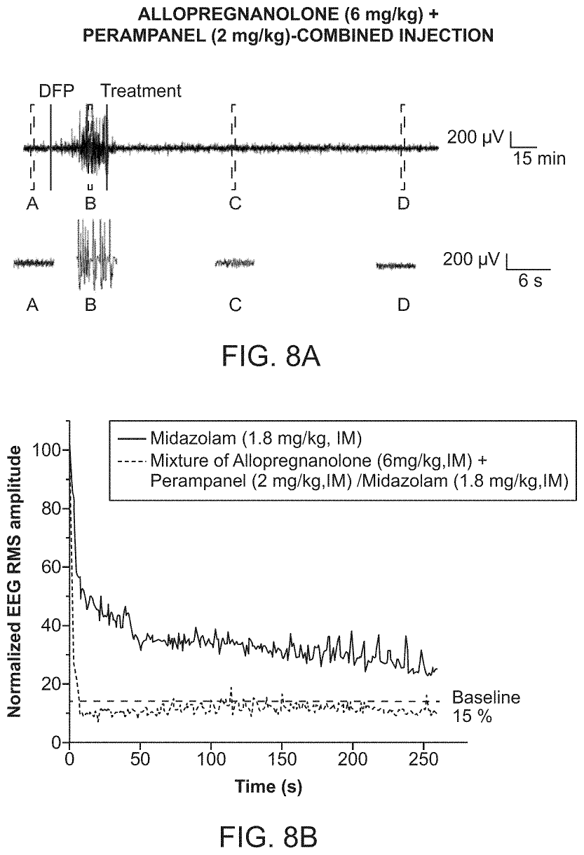

In the second scenario, we injected the mixture of perampanel (2 mg/kg) and allopregnanolone (6 mg/kg) as a single intramuscular injection 40 min after DFP challenge. In order to prepare the mixture, allopregnanolone (6 mg) and perampanel (2 mg) were weighed separately and then combined in a single vial. The mixture was prepared using propylene glycol:ethanol:water in the ratio of 7:2:1. The mixture was prepared with intermittent sonication and warming in order to form a clear solution. Similar to first scenario (employing separately prepared allopregnanolone and perampanel), the standard-of-care midazolam (1.8 mg/kg) was administered as a separate injection. The results of the second scenario are depicted in the FIG. 8.

FIG. 8 shows that the combination of allopregnanolone (6 mg/kg) and perampanel (2 mg/kg) and its administration in rats as a single intramuscular bolus injection along with standard-of-care midazolam resulted in quick cessation of status epilepticus with respect to midazolam (1.8 mg/kg) per se treatment group. As can be seen from FIG. 8, the EEG (top-panel) comes to the baseline when this combined treatment was administered to DFP challenged animals and the effect could be seen within 3-5 min of its administration. The normalized Root Mean Square (RMS) amplitude also shows a quick drop to the baseline (15%) after this treatment. Animals treated with midazolam (1.8 mg/kg, IM) continue to stay in status epilepticus.

The combined effect of allopregnanolone (a neurosteroid) with perampanel (an AMPA receptor antagonist) is superior to allopregnanolone and carbamazepine (a sodium channel standard anti-seizure drug) in DFP status epilepticus animal model. We demonstrate that the combination of a neurosteroid with AMPA receptor antagonist is useful for the effective management of status epilepticus in this animal model. Neuropathology associated with organophosphate poisoning is associated with glutamatergic excitotoxicity. AMPA glutamatergic receptors are involved in the fast synaptic excitation within and between different brain regions and provide a molecular target to manage epilepsy in such cases.

In a subset of animals (n=5-6), we replaced perampanel (an AMPA receptor antagonist) with carbamazepine (a sodium channel blocker) in order to see if there is any change in the efficacy pattern. Carbamazepine was dissolved in a similar vehicle as perampanel, which is propylene glycol:ethanol:water in the ratio of 7:2:1. We have used two treatment groups for this study (A) Allopregnanolone (6 mg/kg, IM) with perampanel (2 mg/kg., IM) (B) Allopregnanolone (6 mg/kg., IM) with carbamazepine (30 mg/kg., IM). Both of these treatment groups received standard-of-care midazolam (1.8 mg/kg., IM). All these injections were administered intramuscular as individual treatments 40 min after DFP challenge.

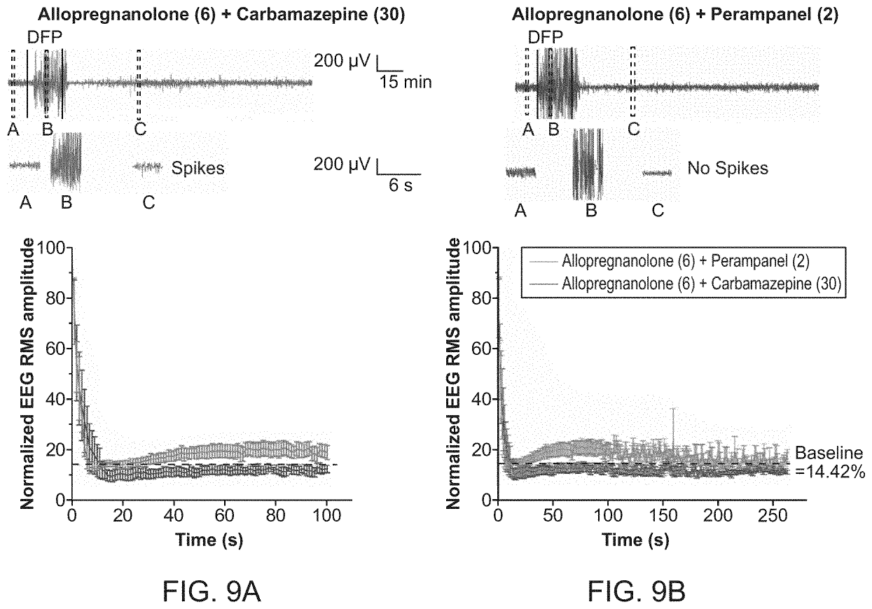

It was seen that although a combination of allopregnanolone and carbamazepine blocked status epilepticus in this animal model; however, the corrected (normalized) EEG power (as RMS value) never came to the baseline (14.42%) (FIG. 9), indicating that there is still some epileptiform activity going into the brain. We can observe some spike activity in these animals after their status is stopped (FIG. 9). In contrast, addition of perampanel with allopregnanolone resulted in quick cessation of status epilepticus, as indicated by EEG returning to the baseline levels. This implies that the addition of an AMPA receptor antagonist is essential in such cases.

Comparison of Allopregnanolone and Perampanel Combination Therapy with Standard Valproate Therapy in the Treatment of Status Epilepticus:

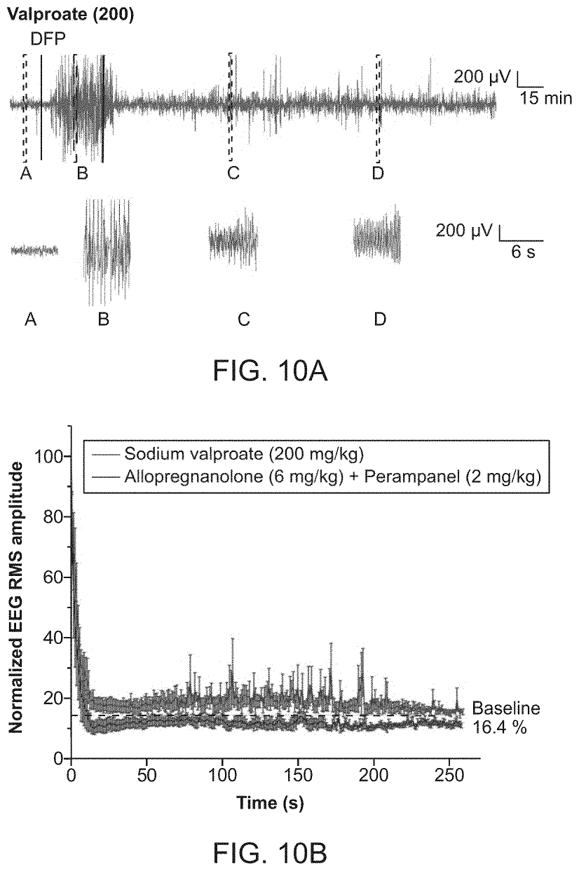

In this experiment, we compared our combination therapy with the standard anti-seizure therapy that is used in clinics for the treatment of status epilepticus. We induced status epilepticus in rats by injecting DFP (4 mg/kg, SC). After 40 min of DFP administration and when the seizures become refractory, animals were given either (A) allopregnanolone (6 mg/kg., IM)+permapanel (2 mg/kg., IM) or (B) Sodium Valproate (200 mg/kg, IP). All these animals were also administered with standard-of-care midazolam (1.8 mg/kg, IM). It was observed that the normalized Root Mean Square amplitude of the sodium valproate group was higher compared to allopregnanolone+perampanel treatment group, demonstrating that allopregnanolone and perampanel combination therapy is far superior than the standard clinical approach. As can be seen from FIG. 10 (top EEG panel), the valproate group shows intermittent spikes and seizure activity during the whole 5 h of EEG recording after DFP administration.

Effect of Perampanel (an AMPA Receptor Antagonist) with the Standard-of Care Midazolam (a Benzodiazepine) in DFP Status Epilepticus.

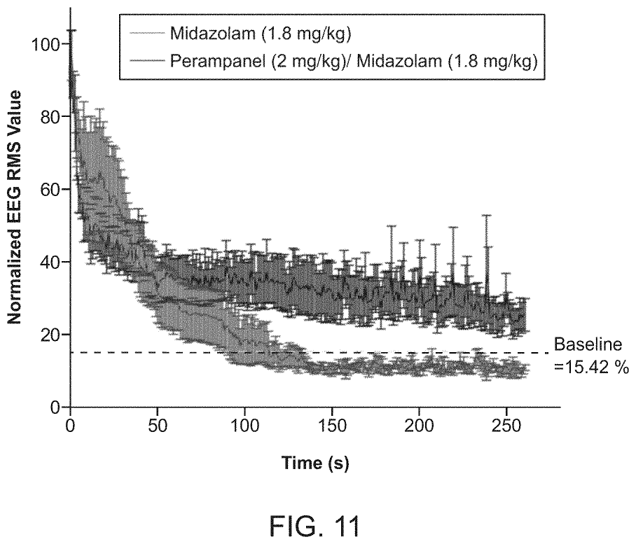

In this protocol, perampanel (2 mg/kg) was administered intramuscularly with standard-of care benzodiazepine (midazolam) in DFP-induced status epilepticus animal model. The treatment was carried out at 40 min after DFP administration and the results were compared with midazolam per se group. It was found that perampanel alone with midazolam did not provide immediate relief from the status epilepticus in this animal model. It took around 90 min to see the difference between two groups (FIG. 11). Therefore, it is concluded that allopregnanolone is really essential if we are targeting quicker relief from status epilepticus.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition comprising a neurosteroid and an α-amino-3-hydroxy-5-methyl-4-isoxazolepropionic acid (AMPA) receptor antagonist.

2. The composition of claim 1, wherein the composition comprises one or more of the neurosteroid and the AMPA receptor antagonist in a subtherapeutic or non-therapeutic dose.

3. The composition of claim 1, further comprising a benzodiazepine.

4. The composition of claim 3, wherein the composition comprises the benzodiazepine in a subtherapeutic dose.

5. The composition of claim 1, wherein the composition is formulated for oral administration.

6. The composition of claim 1, wherein the composition is formulated for parenteral delivery.

7. The composition of claim 6, wherein the parenteral delivery or administration is via a route selected from the group consisting of inhalational, intrapulmonary, intramuscular, subcutaneous, transmucosal and intravenous.

8. The composition of claim 1, wherein the benzodiazepine is a positive modulator of synaptic GABA-A receptors.

9. The composition of claim 1, wherein the benzodiazepine is an agonist of the benzodiazepine recognition site on GABA-A receptors and stimulates endogenous neurosteroid synthesis.

10. The composition of claim 1, wherein the benzodiazepine is selected from the group consisting of bretazenil, clonazepam, cloxazolam, clorazepate, diazepam, fludiazepam, flutoprazepam, lorazepam, midazolam, nimetazepam, nitrazepam, phenazepam, temazepam and clobazam.

11. The composition of claim 1, wherein the benzodiazepine is midazolam.

12. The composition of claim 1, wherein the neurosteroid is a positive modulator of synaptic and extrasynaptic GABA-A receptors.

13. The composition of claim 1, wherein the neurosteroid is selected from the group consisting of allopregnanolone, allotetrahydrodeoxycorticosterone, ganaxolone, alphaxolone, alphadolone, hydroxydione, minaxolone, and Althesin.

14. The composition of claim 1, wherein the neurosteroid is allopregnanolone.

15. The composition of claim 1, wherein the AMPA receptor antagonist is selected from the group consisting of perampanel, selurampanel, talampanel, tezampanel, fanapanel (a.k.a., ZK-200775), irampanel, kynurenic acid, CFM-2, CNQX, CNQX disodium salt, CP 465022 hydrochloride, DNQX, DNQX disodium salt, Evans Blue tetrasodium salt, GYKI 47261 dihydrochloride, GYKI 52466 dihydrochloride, GYKI 53655 hydrochloride, IEM 1925 dihydrobromide, Naspm trihydrochloride, NBQX, NBQX disodium salt, Philanthotoxin 74, SYM 2206, UBP 282, and YM 90K hydrochloride.

16. The composition of claim 1, wherein the AMPA receptor antagonist is a selective antagonist of an AMPA receptor.

17. The composition of claim 1, wherein the AMPA receptor antagonist is perampanel.

18. The composition of claim 1, wherein the composition comprises allopregnanolone and perampanel.

19-31. (canceled)

32. A method of preventing or terminating a seizure in a subject in need thereof, comprising administration to the subject of an effective amount of a composition of claim 1.

33. (canceled)

34. A method of preventing or terminating a seizure in a subject in need thereof, comprising co-administration to the subject of an effective amount of a neurosteroid and an AMPA receptor antagonist.

35-47. (canceled)

48. A method of preventing, treating, reversing, reducing, mitigating and/or ameliorating one or more symptoms associated with a mood disorder or depression in a subject in need thereof, comprising administration to the subject of a an effective amount of the composition of claim 1.

49. A method of preventing, treating, reversing, reducing, mitigating and/or ameliorating one or more symptoms associated with mood disorder or depression in a subject in need thereof, comprising co-administration to the subject of an effective amount of a neurosteroid and an AMPA receptor antagonist.

50-86. (canceled)
